SEQUENCE LISTING (16 sequences)

SEQ ID NO: 1 - DNA, 21 bp, Artificial Sequence
  Synthetic DNA primer
caagagatggccacggctgct

SEQ ID NO: 2 - DNA, 21 bp, Artificial Sequence
  Synthetic DNA primer
tccttctgcatcctgtcggca

SEQ ID NO: 3 - DNA, 18 bp, Artificial Sequence
  Synthetic DNA primer
gagcatgtgagcaaggag

SEQ ID NO: 4 - DNA, 18 bp, Artificial Sequence
  Synthetic DNA primer
aaggaagagtgtcggaag

SEQ ID NO: 5 - DNA, 24 bp, Artificial Sequence
  Synthetic DNA primer
cccaacacggagttctacttgaaa

SEQ ID NO: 6 - DNA, 24 bp, Artificial Sequence
  Synthetic DNA primer
gaagaaatcccgaaacttggtgtc

SEQ ID NO: 7 - DNA, 20 bp, Artificial Sequence
  Synthetic DNA primer
ccatacacagaggcagtcat

SEQ ID NO: 8 - DNA, 20 bp, Artificial Sequence
  Synthetic DNA primer
ggtgtcagatcgatgtcttc

SEQ ID NO: 9 - DNA, 18 bp, Artificial Sequence
  Synthetic DNA primer
cttgtggaggagttgaga

SEQ ID NO: 10 - DNA, 18 bp, Artificial Sequence
  Synthetic DNA primer
tcctgctgagaaaggcat

SEQ ID NO: 11 - DNA, 20 bp, Artificial Sequence
  Synthetic DNA primer
tgatgagaacctgcgcatag

SEQ ID NO: 12 - DNA, 20 bp, Artificial Sequence
  Synthetic DNA primer
accgatgacaggttggtgat

SEQ ID NO: 13 - DNA, 20 bp, Artificial Sequence
  Synthetic DNA primer
agcacaactctgagatatgg

SEQ ID NO: 14 - DNA, 20 bp, Artificial Sequence
  Synthetic DNA primer
atagtcactgtacttgaact

SEQ ID NO: 15 - DNA, 18 bp, Artificial Sequence
  Synthetic DNA primer
ggatgaagaatggaagag

SEQ ID NO: 16 - DNA, 18 bp, Artificial Sequence
  Synthetic DNA primer
tggacatcagggtgagtg

(19) United States
(12) Patent Application Publication
(10) Pub. No.: US 2004/0029153 A1
(43) Pub. Date: Feb. 12, 2004

(54) Method for estimating metabolic function of xenobiotic and induction thereof

(76) Inventors: Junzo Takahashi, Suita-shi (JP); Eiji Aoyama, Osaka-shi (JP); Mitsuhiro Nishihara, Kyoto-shi (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/416,216

(22) PCT Filed: Nov. 16, 2001
(86) PCT No.: PCT/JP01/10015

(30) Foreign Application Priority Data
Nov. 17, 2000 (JP) 2000-351208

(51) Int. Cl. C12Q 1/68; G01N 33/53; G01N 33/567

(52) U.S. Cl. 435/6; 435/7.2

(57) ABSTRACT

Thawed Cryopreserved primary human hepatocytes are maintained in a serum-free synthetic medium containing glucocorticoid, and they are made to contact with a test compound, thereby enabling stable implementation of estimation of metabolic function of xenobiotics and induction thereof, using human hepatocyte retaining the traits for differentiation.

### BACKGROUND OF THE INVENTION

1. Field of the Invention

The present invention relates to (1) cryopreserved primary cultured human hepatocytes, (2) serum-free synthetic medium, (3) a method for maintaining cryopreserved primary cultured human hepatocytes in serum-free synthetic medium, (4) a method for assaying an enzyme activity and a gene expression, involved in xenobiotic metabolism, or the method for inducing the enzyme activity and for inducing the gene expression, involved in xenobiotic metabolism, which is characterized by using the serum-free synthetic medium, (5) a screening method for a compound or a salt thereof that inhibits or enhances induction of the activity or the gene expression of an enzyme involved in xenobiotic metabolism in the liver, which is characterized by using the method, (6) a compound or a salt thereof that inhibits or enhances induction of the activity or the gene expression of an enzyme involved in xenobiotic metabolism in the liver, which is obtained using the screening method, (7) a pharmaceutical composition comprising the compound, (8) a pharmaceutical product or candidate compound thereof whose bioactivity or safety has been verified by using the screening method, (9) a method for determining the effect of a test compound including the pharmaceutical product or candidate compound thereof on the metabolic function of xenobiotics in the liver, which is characterized by the maintenance of the cryopreserved primary cultured human hepatocytes in the serum-free synthetic medium, (10) a method for investigating the effect of a test compound including the pharmaceutical product or candidate compound thereof on the metabolic function of xenobiotics in the liver, which is characterized by the maintenance of the cryopreserved primary cultured human hepatocytes in the serum-free synthetic medium, etc.

2. Description of the Background Art

The liver has numerous physiological functions, and in particular, plays a central role in converting xenobiotics such as pharmaceuticals, food additives and environmental pollutants into excretory forms, so-called xenobiotic metabolism. This function of xenobiotic metabolism may concomitantly cause alterations in mutagenesis by xenobiotics, expression of toxicity and expression of pharmacologic effects. For this reason, studies on metabolism in the liver are indispensable for development of pharmaceuticals and food additives and analysis of environmental pollutants, and studies on the xenobiotic metabolism in the liver have been extensively carried out using experimental animals or hepatocytes obtained from experimental animals. However, it is well known that activities of enzymes involved in so-called xenobiotic metabolism in the liver are different between human and experimental animals such as mice, rats, rabbits and monkeys both in quality and in quantity, and that the findings obtained by using experimental animals often fail to apply to human (J. C. Merrill, D. J. Beck, D. A. Kaminski, A. P. Li, Toxicology 99(3), 147-152 (1995)). Thus, it is essential to use human livers or the cells derived from human liver to have proper understanding of the influence of xenobiotics on the human hepatic metabolism or xenobiotic metabolism in the human liver. To carry out such experiments, it may be possible to administer a test compound to volunteers, but there might be not a few risks of serious damage resulting from an unexpected effect of the test compound or its metabolite on human subjects. Therefore, it is considered effective to use hepatocytes isolated from the tissues dissected from living bodies in the experiments, but it is impossible to subculture (i.e. infinitely increase the cell number ex vivo) primary hepatocytes isolated from living organisms. On the other hand, those cells which are allowed to be established as cell lines with theoretical capability of infinite proliferation often lose their inherent traits for differentiation, and they often fail to precisely reflect the functions of the tissues from which they are derived. In particular, in hepatocytes, enzymes involved in xenobiotic metabolism easily lose their activities even in their primary cultures. To date, it is found that no established cell lines sufficiently retain those traits (J. Dich et al., Hepatology, 8(1), 39-45 (1988)). Therefore, human hepatocytes that retain the metabolic capacity of xenobiotics and can be stably maintained during the experiments have been widely sought so far. Hitherto, there was a report on an actual study using primary hepatocytes that were obtained from a fresh section of liver extirpated during surgery or fresh liver extirpated from a brain-death patient, or the liver removed by treating them using a well known method of perfusion with collagenase (L. Pichard-Garcia et al., Drug Metabolism & Disposition 28(1), 51-57 (2000)). In such cases, hepatocytes should be prepared immediately after the liver was removed from the patient. There are many disadvantages in utilization of such cells when they are industrially used, since fresh human liver available for research purposes is extremely limited in number, explicit consent of the donors is required for such use, and exact date and time of acquisition of such tissue. Moreover, even if fresh human liver available for research purposes is actually obtained, ethical issues will prevent researchers from using it freely. In addition, some donors might be infected with dangerous virus, such as HIV, HBV and HCV, which could cause serious disorders, and it is required to verify that the liver is not contaminated with such dangerous virus before the fresh liver is used for research purposes. Furthermore, not a few enzymes responsible for xenobiotic metabolism in the hepatocyte exhibit greatly differences in activity level among individuals (T. Shimada et al., The Journal of Pharmacology and Experimental Therapeutics 270(1), 414-423 (1994)). Accordingly, it is desired to carry out an experiment using fresh livers from several donors in order to obtain reliable measurements, but in fact, it is almost impossible to observe variation among individuals by simultaneously using fresh livers obtained from several donors whose safety has been confirmed. To overcome this difficulty, the studies aiming for utilizing human hepatocytes cryopreserved in the liquid nitrogen have been carried on. Although cryopreserved human hepatocytes have been commercially available in recent years, their viability and stability in the function to xenobiotic metabolism are often severely impaired when the cryopreserved primary cells are used. Also, expression of a number of liver functions in primary hepatocytes is largely affected by various sorts of ingredients and supplements such as serum, contained in the medium (R. P. Evarts, E. Marsden, S. S. Thorgeirsson, Biochemical Pharmacology 33(4), 565-569 (1984)). As described above, there still remain a lot of problems in stably utilizing cryopreserved human primary hepatocytes for the research purposes. Although the enzyme activity involved in xenobiotic metabolism and the gene expression, or induction of the enzyme activity involved in xenobiotic metabolism and the gene expression were not previously reported to have been observed, a technique which allows such observation is extensively required.

For the reasons described above, if cryopreserved primary human hepatocytes of several donors whose safety was confirmed can be maintained in the serum-free synthetic medium, and the enzyme activity and the gene expression, involved in xenobiotic metabolism, and induction of the enzyme activity and the gene expression, involved in xenobiotic metabolism can be measured stably, it may be industrially very beneficial and greatly useful for developing pharmaceuticals that act on liver functions including xenobiotic metabolism and for the studies on influences of pharmaceuticals, food additives and environmental pollutants on the human bodies, including safety and pharmacologic effects.

### OBJECTS OF THE INVENTION

An object of the present invention is to develop a technique for maintaining cryopreserved primary human hepatocytes, which retain their traits as liver, by the serum-free synthetic medium, and stably measuring the enzyme activity and the gene expression, involved in liver functions, in particular xenobiotic metabolism, or induction of the enzyme activity and of the gene expression, involved in xenobiotic metabolism, thereby enabling the development of pharmaceuticals that act on liver functions such as xenobiotic metabolism, and the studies on influences of pharmaceuticals, food additives and environmental pollutants on the human bodies, including safety and pharmacological effects.

### SUMMARY OF THE INVENTION

In view of the aforementioned object, the present inventors extensively studied, and finally have established a technique that allows maintenance of cryopreserved primary human hepatocytes by using serum-free synthetic medium as well as stably measuring, among liver functions, in particular the enzyme activity and the gene expression, involved in xenobiotic metabolism, and induction of the enzyme activity involved in xenobiotic metabolism and of the gene expression. As a result of further study, the present inventors have completed the present invention.

That is, the present invention provides:

(1) a method for assaying the function of a test compound to metabolize xenobiotics or the induction thereof which comprises contacting the test compound with hepatocytes maintained in a serum-free synthetic medium containing glucocorticoid, wherein the hepatocytes are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism;

(2) the method according to the above (1), wherein glucocorticoid is hydrocortisone, dexamethasone or a mixture thereof;

(3) the method according to the above (1), wherein glucocorticoid is hydrocortisone;

(4) the method according to any one of the above (1) to (3), wherein the enzyme is UDP-glucuronyl transferase, flavin-containing monooxygenase, epoxide hydrolase, sulfotransferase, glutathione S-transferase, NADPH-cytochrome P450 reductase or cytochrome P450;

(5) the method according to the above (4), wherein cytochrome P450 is CYP1A1, CYP1A2, CYP2A6, CYP2B6, CYP2C8, CYP2C9, CYP2C19, CYP2D6, CYP2E1, CYP3A4, CYP3A5 or CYP3A7;

(6) the method according to any one of the above (1) to (3), wherein the activity of the enzyme is that of UDP-glucuronyl transferase, flavin-containing monooxygenase, epoxide hydrolase, sulfotransferase, glutathione S-transferase, NADPH-cytochrome P450 reductase, methoxyresorfin dealkylation, ethoxyresorfin dealkylation, pentoxyresorfin dealkylation, benzyloxyresorfin dealkylation, ethoxycoumarin dealkylation, coumarin hydroxylation, taxol hydroxylation, tolbutamide hydroxylation, (S)-mephenytoin hydroxylation, bufuralol hydroxylation, nitrophenol hydroxylation or testosterone hydroxylation;

(7) the method according to any one of the above (1) to (3) which measures the enzyme activity or the gene expression, involved in xenobiotic metabolism;

(8) the method according to any one of the above (1) to (3) which measures the mechanism for inducing the enzyme activity or the mechanism for inducing the gene expression, involved in xenobiotic metabolism;

(9) the method according to any one of the above (1) to (3), wherein the serum-free synthetic medium further comprises one or more components selected from the group consisting of prolactin (P), cholera toxin (C) and liver cell growth factor (LCGF) (L) (i.e. containing any one of (i) P, (ii) C, (iii) L, (iv) P and C, (v) P and L, (vi) C and L, or (vii) P, L and C);

(10) a method for maintaining (i) the enzyme activity or the gene expression, involved in xenobiotic metabolism, or (ii) the mechanism for inducing the enzyme activity or the mechanism for inducing the gene expression, involved in xenobiotic metabolism of hepatocytes, which comprises culturing cryopreserved primary human hepatocytes in a serum-free synthetic medium containing glucocorticoid after the hepatocytes are thawed;

(11) the method according to the above (10), wherein glucocorticoid is hydrocortisone, dexamethasone or a mixture thereof;

(12) the method according to the above (10), wherein glucocorticoid is hydrocortisone;

(13) the method according to any one of the above (10) to (12), wherein the serum-free synthetic medium further comprises one or more components selected from the group consisting of prolactin, cholera toxin and liver cell growth factor (LCGF);

(14) hepatocytes maintained by the method according to any one of the above (10) to (12);

(15) a serum-free synthetic medium for culturing cryopreserved primary human hepatocytes after thawing which comprises glucocorticoid, prolactin, cholera toxin and liver cell growth factor (LCGF);

(16) the serum-free synthetic medium according to the above

(15), wherein glucocorticoid is hydrocortisone, dexamethasone or a mixture thereof;

(17) the serum-free synthetic medium according to the above

(15), wherein glucocorticoid is hydrocrotisone;

(18) a method for screening for a compound or a salt thereof that inhibits or enhances (or has no effect on) (i) the enzyme activity or the gene expression, involved in xenobiotic metabolism in the liver, or (ii) the mechanism for inducing the enzyme activity or the mechanism for inducing the gene expression, involved in xenobiotic metabolism in the liver, which comprises using the method according to any one of the above (1) to (3);

(19) a compound or a salt thereof that inhibits or enhances (i) the enzyme activity or the gene expression, involved in xenobiotic metabolism in the liver, or (ii) the mechanism for inducing the enzyme activity or for inducing the gene expression, involved in xenobiotic metabolism in the liver, which is obtained by the screening method according to the above (18);

(20) a pharmaceutical composition comprising the compound or the salt thereof according to the above (19);

(21) a method for determining the effect of a test compound on the function of the liver to metabolize xenobiotics, which comprises using the method according to any one of the above (1) to (3);

(22) use of a serum-free synthetic medium containing glucocorticoid for assaying the function of a test compound to metabolize xenobiotics or the induction thereof by contacting the test compound with hepatocytes which are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism;

(23) use of glucocorticoid for preparing a serum-free synthetic medium which is used for assaying the function of a test compound to metabolize xenobiotics or the induction thereof by contacting the test compound with hepatocytes which are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism;

(24) use of hepatocytes which are obtained by thawing cryopreserved primary human hepatocytes and retain (i) the enzyme activity or the gene expression, involved in xenobiotic metabolism, or (ii) the mechanism for inducing the enzyme activity or for inducing the gene expression, involved in xenobiotic metabolism, for assaying the function of a test compound to metabolize xenobiotics or the induction thereof by contacting the test compound with the hepatocytes maintained in a serum-free synthetic medium containing glucocorticoid;

(25) a method for testing the effect of a test compound containing a pharmaceutical product or a candidate compound thereof on the function of the liver to metabolize xenobiotics which comprises using the method according to the above (1);

(26) a pharmaceutical product or a candidate compound thereof which is shown to have a physiological activity and to be highly safe using the method according to the above (1), and the like.

### BRIEF DESCRIPTION OF THE DRAWINGS

FIG. 1 shows western blotting that demonstrates induction of CYP1A protein by 3-methylcholanthrene and benz[a]anthracene in primary human hepatocytes.

In FIG. 2, FIG. 2A shows western blotting using anti-human CYP3A4 goat IgG, which demonstrates induction of CYP3A protein by rifampicin and phenobarbital in primary human hepatocytes; and FIG. 2B shows western blotting using anti-human CYP3A5 goat IgG, which demonstrates induction of CYP3A protein by rifampicin and phenobarbital in primary human hepatocytes.

FIG. 3 shows polyacrylamide gel electrophoresis that demonstrates expression of various CYP genes in primary human hepatocytes.

FIG. 4 shows polyacrylamide gel electrophoresis that demonstrates differentiation between CYP1A1 and CYP1A2.

FIG. 5 shows polyacrylamide gel electrophoresis that demonstrates differentiation among CYP2C8 and 2C9 and 2C19.

FIG. 6 shows polyacrylamide gel electrophoresis that demonstrates differentiation among CYP3A4 and 3A5 and 3A7.

FIG. 7 is a graph showing the effect of the maintenance method of primary hepatocytes on the induction of testosterone hydroxylation activity.

FIG. 8 is a graph showing the effect of the ingredients of the medium on the induction of testosterone hydroxylation activity in the primary hepatocytes.

FIG. 9 is a graph showing the effect of the use of serum when seeding the primary human hepatocytes on the induction of testosterone hydroxylation activity in the hepatocytes.

FIG. 10 is a graph showing changes in testosterone hydroxylation activity after induction by chemical agents with time.

FIG. 11 is a graph showing individual difference in the testosterone hydroxylation activity and its induction.

FIG. 12 is a graph showing individual difference in the amount of CYP3A mRNA and its induction.

FIG. 13 is a graph showing changes in ethoxyresorfin dealkylation activity after induction by chemical agents with time.

FIG. 14 is a graph showing individual difference in the ethoxyresorfin dealkylation activity and its induction.

FIG. 15 is a graph showing individual difference in the amount of CYP1A mRNA and its induction.

In FIG. 16, FIG. 16A is a graph showing changes in ethoxycoumarin dealkylation and conjugation activities in HH-110 by benz[a]anthracene and 3-methylcholanthrene; and FIG. 16B is a graph showing changes in ethoxycoumarin dealkylation and conjugation activities in HH-118 by benz[a]anthracene and 3-methylcholanthrene.

FIG. 17 is a graph showing concentration-dependence of induction of testosterone hydroxylation activity by various CYP3A inducing agents.

FIG. 18 is a graph showing concentration-dependence of CYP3A mRNA induction by various CYP3A inducing agents.

FIG. 19 is a graph showing concentration-dependence of induction of ethoxyresorfin dealkylation activity by 3-methylcholanthrene and benz[a]anthracene.

FIG. 20 is a graph showing concentration-dependence of CYP1A mRNA induction by 3-methylcholanthrene and benz[a]anthracene.

FIG. 21 is a graph showing testosterone hydroxylation activity of the primary heptocytes purchased from Tissue Transformation Technologies, Inc. (MD, USA), In Vitro Technologies, Inc. (MD, USA) and XenoTech, LLC (KS, USA).

FIG. 22 is a graph showing the effect of the concentration of hydrocortisone on the testosterone hydroxylation activity.

FIG. 23 shows a graph showing the effect of 3 kinds of glucocorticoid.

FIG. 24 is a graph showing the effect of hydroxy group of hydrocortisone on the testosterone hydroxylation activity and structures of hydrocortisone analogue used.

### DETAILED DESCRIPTION OF THE INVENTION

As used herein, “to metabolize xenobiotics” or “xenobiotic metabolism” means metabolism of, for example, pharmaceuticals, food additives and environmental pollutants, inter alia, drug metabolism and the like are preferably used. For human hepatocytes, cells obtained from normal tissue, including a fresh section of liver partially excised from a human adult during surgery and fresh liver excised from a brain-death patient, and the excised liver, by treating them using a well-known method, such as perfusion with collagenase (A. P. Li et al., J. Tiss, Cult. Meth. 14, 139-146 (1992)). The so-called primary hepatocytes thus obtained were dispersed in the cell cultured medium containing 5-20% dimethylsulfoxide and 5-20% fetal bovine serum, or commercially available solution for freeze-preservation of cells, such as Cellbanker (“serubankar”, Nippon Zenyaku Kogyo Co., Ltd.) and Cellvation (CELOX Corporation), and the cells were frozen according to a well known method, such as using a program freezer. The cells thus frozen can be stored in the stable state for more than several years in the liquid nitrogen or in the nitrogen gas phase cooled below −140° C. with liquid nitrogen (A. Ostrowska et al., Cell and Tissue Banking, 1. 55-68 (2000)).

The cells thus preserved can be maintained if necessary, after thawed again. Generally, the cells are thawed rapidly at 37° C., and, if necessary, washed 1-5 times with MEM medium (H. Eagle, Science 130, 432-437 (1959)), DMEM medium (R. Dulbecco and G. Freeman, Virology 8, 396-397 (1959)), Williams' E medium (G. M. Williams and J. M. Gunn, Exp. Cell. Res. 89, 139-142 (1974)), Leibovitz's L-15 medium (L-15 medium) (A. Leibovitz, Am. J. Hyg. 78, 173-180 (1963)), Landford's medium (R. E. Lanford et al., In Vitro Cellular & Developmental Biology 25(2), 174-182 (1989)) and the like, which cooled to 4° C. Subsequently, the cells are desirably maintained one day and night in any of the media mentioned above or the like which contains 5-20% fetal bovine serum. When the survival rate is low, the cells whose relative density has been reduced due to damage can be removed during washing by using higher-density washing medium containing, for example, sucrose or Percoll (Amersham Pharmacia Biotech KK.). From the cells thus obtained, cells are selected which retain the enzyme activity or the gene expression, involved in xehobiotic metabolism, or the mechanism for inducing the enzyme activity or for inducing the gene expression, involved in xenobiotic metabolism. Subsequently, the cells are maintained in a serum-free synthetic medium (e.g. Landford's medium) containing glucocorticoid as an essential component, using a well known culturing method and the like. Glucocorticoid is added to the medium at a concentration of 1 nmol/L to 100 μmol/L, particularly, in case of using hydrocortisone, preferably at a concentration of 1 μmol/L to 10 μmol/mL. Furthermore, any one component selected from, preferably two components selected from, and more preferably all the three components from the group consisting prolactin, cholera toxin and liver cell growth factor (LCGF) may be added to the medium. The contents of these components are 100 μg/L for prolactin, 2 μg/L for cholera toxin, and 5 mg/L for liver cell growth factor (LCGF). The cells are maintained preferably in the incubator saturated with moisture vapor containing 5% carbon dioxide. Preferably, pH is approximately 6.5-7.5 and temperature is around 37° C. Preferably, culture vessels are treated with a substance that facilitates cell adhesion (e.g. collagen, collagen gel, MATRIGEL, etc.). Alternatively, carriers for cell culture, such as collagen sponge, may be used. Above all, a 12-well culture plate coated with collagen is preferably used. On culturing, preferably 5-10 millions of cells per well are seeded. Medium is preferably replaced with fresh medium 8 to 24 hours after seeding, after that the medium is replaced with fresh medium every 24 to 72 hours. From the cells thus maintained, cells adhered to culture vessels are preferably used.

As used herein, “glucocorticoid” means, among adrenocortical hormones, steroids relating to carbohydrate metabolism (e.g., cortisol, corticosterone, cortisone, hydrocortisone, etc.) and synthetic materials having similar activities (e.g., dexamethasone, predonisolone, etc.). In the present invention, these steroids and synthetic materials can be used alone or in a combination of two or more thereof. Among them, as “glucocorticoid”, preferred are hydrocortisone, dexamethasone or a mixture of hydrocortisone and dexamethasone, in particular, hydrocortisone.

Enzymes involved in liver-specific xenobiotic metabolism include UDP-glucuronyl transferase, flavin-containing monooxygenase, epoxide hydrolase, sulfotransferase, glutathione S-transferase, NADPH-cytochrome P450 reductase, cytochrome P450 and the like. Among them, cytochrome P450 is the most important enzyme group in terms of their distribution and functions in xenobiotic metabolism.

Cytochrome P450 is a general name for a large number of enzyme proteins. As individual names of cytochrome P450 involved in xenobiotic metabolism in the liver, CYP1A1, CYP1A2, CYP2A6, CYP2B6, CYP2C (particularly, CYP2C8, CYP2C9 and CYP2C19), CYP2D6, CYP2E1, CYP3A (particularly, CYP3A4, CYP3A5 and CYP3A7) and so forth are known. Among them, CYP1A (particularly, CYP1A1 or CYP1A2), CYP3A (particularly, CYP3A4 or CYP3A5) and so forth are preferably used. Presence of NADPH-cytochrome P450 reductase is required for activation of cytochrome P450. Also, a large number of xenobiotic metabolizing enzymes are known to be induced under certain conditions. For instance, effect of so-called polycyclic aromatics, such as benzo[a]pyrene, benz[a]anthracene, 3-methylcholanthrene, dioxin, on CYP1A expression, effect of phenobarbital and phenobarbitone on CYP2B expression, and effect of rifampicin, dexamethasone phenytoin and phenylbutazone on CYP3A expression are well known (C. G. Gibson et al., New Metabolomics of Xenobiotics, Kodansha Ltd. (1995)).

Enzymatic activities involved in liver-specific metabolism of xenobiotics, include, for example, the activities of UDP-glucuronyl transferase, flavin-containing monooxygenase, epoxide hydrolase, sulfotransferase, glutathione S-transferase, and mixed function oxidase (MFO) composed of NADPH-cytochrome P450 reductase and cytochrome P450 (e.g., methoxyresorfin dealkylation, ethoxyresorfin dealkylation, pentoxyresorfin dealkylation, benzyloxyresorfin dealkylation, ethoxycoumarin dealkylation, coumarin hydroxylation, taxol hydroxylation, tolbutamide hydroxylation, (S)-mephenytoin hydroxylation, bufuralol hydroxylation, nitrophenol hydroxylation and testosterone hydroxylation activities, etc.). Among them, UDP-glucuronyl transferase, flavin-containing monooxygenase and MFO activities are important, in particular, cytochrome P450 activity that is detectable as MFO activity is considered as the most important enzymatic activity with regard to, for example, the function involved in xenobiotic metabolism.

Since cryopreserved primary human hepatocytes are able to maintain their liver functions involved in xenobiotic metabolism in the present invention, the activity or expression of the liver-specific enzymes, as mentioned above, that are involved in xenobiotic metabolism, the induction of the activity or the gene expression of the liver-specific enzymes that are involved in xenobiotic metabolism (preferably the both), and such can be determined. This determination can be used in various methods such as for screening for a compound that shows therapeutic and prophylactic effects on the diseases associated with aberration in xenobiotic metabolism in the liver (e.g. liver dysfunction); for investigating effects of pharmaceuticals and candidate pharmaceutical compounds on the xenobiotic metabolism in the liver; and for detection of the effects of pharmaceuticals and candidate pharmaceutical compounds on the functions of xenobiotic metabolism. Thus, the present invention provides a method of screening for a compound or a salt thereof that inhibits or enhances (or has no effect on) the enzyme activity or the gene expression, involved in xenobiotic metabolism in the liver, or the mechanism for inducing the enzyme activity or the mechanism for inducing the gene expression, involved in xenobiotic metabolism, via the technique of the present invention, by contacting a test compound with cryopreserved primary cultured human hepatocytes that retain the enzyme activity or the gene expression, involved in xenobiotic metabolism, or the mechanism for inducing the enzyme activity or the mechanism for inducing the gene expression, involved in xenobiotic metabolism; a method for investigating the effects of a test compound containing a pharmaceutical or candidate pharmaceutical compound on the liver functions for xenobiotic metabolism; and a compound or a salt thereof obtained by said screening method; a pharmaceutical composition comprising said compound or the salt form thereof; etc. The present invention also provides a method for maintaining in the hepatocytes (i) the enzyme activity or the gene expression, involved in xenobiotic metabolism, or (ii) the mechanism for inducing the enzyme activity or the mechanism for inducing the gene expression, involved in xenobiotic metabolism, which comprises culturing cryopreserved primary human hepatocytes in serum-free synthetic medium containing glucocorticoid after the hepatocytes are thawed; hepatocytes maintained using the method; the serum-free synthetic medium for culturing cryopreserved primary human hepatocytes after thawed, which comprises glucocorticoid and further one or more components selected from the group consisting of prolactin, cholera toxin and liver cell growth factor (LCGF); etc. Herein, preferred contents of glucocorticoid, prolactin, cholera toxin and liver cell growth factor (LCGF) are as described above. By the term “synthetic medium” is meant that the components contained in the medium are all already identified substances (i.e. the medium is free of unidentified substances).

Test compounds include, for example, peptides, proteins, nonpeptidic natural products, synthetic compounds, fermented products, cell extracts, plant extracts, animal tissue extracts, plasma and the like. These compounds may be novel compounds or known compounds. Specifically, the method of the present invention can be used to study or examine a test compound for its therapeutic and prophylaxis effects and its effects on the liver functions for xenobiotic metabolism with the guidance of the enzyme activity or the gene expression involved in xenobiotic metabolism, or the mechanism for inducing the enzyme activity or the gene expression involved in xenobiotic metabolism of cryopreserved primary human hepatocytes by treating cryopreserved primary human hepatocytes that stably maintain the enzyme activity or the gene expression involved in xenobiotic metabolism, or the mechanism for inducing the enzyme activity or the gene expression involved in xenobiotic metabolism with the test compound to compare with untreated controls.

A compound obtained by using the screening or determination method of the present invention is selected from the test compounds described above, and it can be used as (1) a pharmaceutical having a therapeutic and prophylactic effects on the diseases associated with aberration in xenobiotic metabolism in the liver (e.g. liver dysfunction), (2) a pharmaceutical less toxic to the liver, or (3) a safe and less toxic pharmaceutical, such as a therapeutic and prophylaxis against the diseases, since the effect of the compound on the metabolism in the liver has been confirmed. Likewise, compounds derivatized from the compounds obtained by the aforementioned screening or determination method can be used. A test concentration of a test compound preferably ranges from approximately 1 nmol/L to 1 mol/L. The compounds may be used in the form of solutions in which a test compound is dissolved in a solvent, such as physiological saline, methanol and dimethylsulfoxide. The percentage of such solvent in the medium is preferably 0.1% to 1%. A compound obtained using the screening or determination method may be in the salt form. The salt forms of the compounds include salts with physiological acceptable acids (e.g. inorganic acids and organic acids) or bases (e.g. alkaline metals), inter alia, physiologically acceptable acid-added salts are preferable. Such salts include, for example, salts with inorganic acids (e.g. hydrochloric acid, phosphoric acid, hydrobromic acid and sulfuric acid) or organic acids (e.g. acetic acid, formic acid, propionic acid, fumaric acid, maleic acid, succinic acid, tartaric acid, citric acid, malic acid, oxalic acid, benzoic acid, methansulfonic acid and benzensulfonic acid).

The compound or a salt thereof obtained by the screening or determination method (hereinafter sometimes referred to as “the compound of the present invention”) may be prodrugs or a hydrate. Herein, a prodrug is any compound that is converted into the compound of the present invention through the reaction of enzyme, gastric acid or the like under the physiological conditions in vivo, i.e. a compound converted into the compound of the present invention by enzymatic oxidation, reduction, hydrolysis and the like, or a compound converted into the compound of the present invention by hydrolysis by gastric acid, etc.

Prodrugs of the compound of the present invention include a compound in which an amino group of the compound of the present invention is acylated, alkylated or phosphorylated (e.g. a compound in which an amino group is eicosanoylated, alanylated, pentylaminocarbonylated, (5-methyl-2-oxo-1,3-dioxolene-4-yl) methoxycarbonylated, tetrahydrofuranylated, pyrrolidylmethylated, pivaloyloxymethylated, tert-butylated, etc.); a compound in which a hydroxyl group is acylated, alkylated, phosphorylated or borated (e.g. a compound in which an hydroxyl group is acetylated palmitoylated, propanoylated, pivaloylated, succinylated, fumarylated, alanylated, dimethylaminomethylcarbonylated, etc.); a compound in which a carboxyl group is esterified or amidated (e.g. a compound in which a carboxyl group is ethylesterified, phenylesterified, carboxymethylesterified, dimethylaminomethylesterified, pivaloyloxymethylesterified, ethoxycarbonyloxyethylesterified, phthalidylesterified, (5-methyl-2-oxo-1,3-dioxolene-4-yl) methylesterified, cyclohexyloxycarbonylethylesterified, methylamidated, etc.). These compounds can be produced from the compound of the present invention, using a well know method per se.

A prodrug of the compound of the present invention may be converted into the compound of the present invention under physiological conditions such as described in “Development of pharmaceutical Products Vol. 7 Molecular Design”, pp. 163-198, Hirokawa Shoten Inc. (1990).

A pharmaceutical composition containing the compound or a salt thereof obtained using the screening or determination method can be produced, using the above compound of the present invention or a salt thereof and according to a well known method per se.

The pharmaceutical composition of the present invention may contain the compound of the present invention, and a pharmacologically acceptable carrier, diluent or vehicle. This composition is provided in a dosage form suitable for oral or parenteral administration.

Thus, compositions to be administered orally, for example, include solid and liquid dosage forms, specifically, tablets (including sugar- or film-coated tablets), balls, granules, powders, capsules (including soft-capsules), syrups, emulsions, suspensions, etc. These compositions can be produced by a well-known method per se, and contain a carrier, diluent and excipient generally used in the field of pharmaceutical. For instance, carriers and excipients for tablets include lactose, starch, sucrose, magnesium stearate, etc.

Compositions for parenteral administration are used as injections, suppositories, etc. Injections include dosage forms of intravenous, subcutaneous, intracutaneous, intramuscular injections, drip infusion, etc. These injections are prepared, according to any method well known per se, for example, by dissolving, suspending or emulsifying the compound of the present invention in a sterilized aqueous or oily solution that is usually used as an injection. Aqueous solutions used for injection include, for example, physiological saline, isotonic solution supplemented with glucose and others, which can be used with appropriate solubilizing agents, such as alcohols (e.g. ethanol), polyalcohols (e.g. propyleneglycol, polyethyleneglycol), nonionic detergents (e.g. Polysolbate 80, HCO-50 (polyoxyethylene (50 mol) adduct of hydrogenated castor oil)), etc. Oily solutions used for injection include, for example, sesami oil and soybean oil, which can be used with solubilizing agents, such as benzyl benzoate and benzyl alcohol, etc. Injections prepared are usually filled in appropriate ampoules. Suppositories used for rectal administration are prepared by mixing the aforementioned compound with a conventional suppository base.

The oral or parenteral pharmaceutical compositions are advantageously formulated into any dosage forms that can accommodate the dose of active ingredient. Such dosage forms are exemplified by tablets, balls, capsules, injections (ampoules), suppositories, etc. For each dosage, generally 0.1-100 mg of the aforementioned compound, in particular, 1-50 mg for injections and 1-100 mg for other dosage forms, is preferably contained.

The pharmaceutical compositions thus obtained are safe and less toxic, and they can be administered to, for example, humans or mammals (e.g. rats, mice, guinea pigs, rabbits, sheep, pigs, cattle, horses, cats, dogs, monkeys, etc.). Dosage of the compound or its salt will vary depending on the target disease, subject to be administered, and route of administration. For instance, in case where the compound is orally administered for therapeutic purpose of treating liver dysfunction, typically approximately 0.1-100 mg per day, preferably, approximately 1.0-50 mg per day, more preferably, approximately 1.0-20 mg of the compound per day is administered to an adult (based on a body weight of 60 kg). When administered parenterally, the dose of the compound will vary depending on the subject to be administered and target disease. For instance, in case where the compound is parenterally administered for therapeutic purpose of treating liver dysfunction, approximately 0.01-30 mg, preferably, approximately 0.1-20 mg, more preferably, approximately 0.1-10 mg of the compound per day is advantageously administered intravenously to an adult (based on a body weight of 60 kg). For other animals, doses converted to a body weight of 60 kg can be administered.

When bases and others are denoted by symbols herein, such symbols correspond to the symbols according to IUPAC-IUB Commission on Biochemical Nomenclature, or they are based on the conventional denotations. Some examples are shown below:

A: adenine

T: thymine

G: guanine

C: cytosine

Sequence ID numbers assigned herein represent the sequences indicated below:

[SEQ ID No. 1]

Representing the base sequence of the synthetic forward primer for β-actin that was used in the RT-PCR performed in Examples 6, 11, 13, 15 and 16 described below,

Sequence: caagagatggccacggctgct;

[SEQ ID No. 2]

Representing the base sequence of the synthetic reverse primer for β-actin that was used in the RT-PCR performed in Examples 6, 11, 13, 15 and 16 described below,

Sequence: tccttctgcatcctgtcggca;

[SEQ ID No. 3]

Representing the base sequence of the synthetic forward primer for CYP1A1 and CYP1A2 that was used in the RT-PCR performed in Examples 6, 7, 13, and 16 described below,

Sequence: gagcatgtgagcaaggag;

[SEQ ID No. 4]

Representing the base sequence of the synthetic reverse primer for CYP1A1 and CYP1A2 that was used in the RT-PCR performed in Examples 6, 7, 13, and 16 described below,

Sequence: aaggaagagtgtcggaag;

[SEQ ID No. 5]

Representing the base sequence of the synthetic forward primer for CYP2A6 that was used in the RT-PCR performed in Example 6 described below,

Sequence: cccaacacggagttctacttgaaa;

[SEQ ID No. 6]

Representing the base sequence of the synthetic reverse primer for CYP2A6 that was used in the RT-PCR performed in Example 6 described below,

Sequence: gaagaaatcccgaaacttggtgtc;

[SEQ ID No. 7]

Representing the base sequence of the synthetic forward primer for CYP2B6 that was used in the RT-PCR performed in Example 6 described below,

Sequence: ccatacacagaggcagtcat;

[SEQ ID No. 8]

Representing the base sequence of the synthetic reverse primer for CYP2B6 that was used in the RT-PCR performed in Example 6 described below,

Sequence: ggtgtcagatcgatgtcttc;

[SEQ ID No. 9]

Representing the base sequence of the synthetic forward primer for CYP2C8, CYP2C9 and CYP2C19 that was used in the RT-PCR performed in Examples 6 and 7 described below,

Sequence: cttgtggaggagttgaga;

[SEQ ID No. 10]

Representing the base sequence of the synthetic reverse primer for CYP2C8, CYP2C9 and CYP2C19 that was used in the RT-PCR performed in Examples 6 and 7 described below,

Sequence: tcctgctgagaaaggcat;

[SEQ ID No. 11]

Representing the base sequence of the synthetic forward primer for CYP2D6 that was used in the RT-PCR performed in Example 6 described below,

Sequence: tgatgagaacctgcgcatag;

[SEQ ID No. 12]

Representing the base sequence of the synthetic reverse primer for CYP2D6 that was used in the RT-PCR performed in Example 6 described below,

Sequence: accgatgacaggttggtgat;

[SEQ ID No. 13]

Representing the base sequence of the synthetic forward primer for CYP2E1 that was used in the RT-PCR performed in Example 6 described below,

Sequence: agcacaactctgagatatgg;

[SEQ ID No. 14]

Representing the base sequence of the synthetic reverse primer for CYP2E1 that was used in the RT-PCR performed in Example 6 described below,

Sequence: atagtcactgtacttgaact;

[SEQ ID No. 15]

Representing the base sequence of the synthetic forward primer for CYP3A4, CYP3A5 and CYP3A7 that was used in the RT-PCR performed in Examples 6, 7, 11 and 15 described below,

Sequence: ggatgaagaatggaagag; and

[SEQ ID No. 16]

Representing the base sequence of the synthetic reverse primer for CYP3A4, CYP3A5 and CYP3A7 that was used in the RT-PCR performed in Examples 6, 7, 11 and 15 described below,

Sequence: tggacatcagggtgagtg.

The present invention is further illustrated in detail in the following examples, which are not intended to limit the scope of the invention.

### EXAMPLE 1

### Production and Maintenance of Cryopreserved Primary Human Hepatocytes

Cryopreserved primary human hepatocytes prepared from five different donors were purchased from Tissue Transformation Technologies (NJ, USA). Information on the donors is shown below as hepatocyte donors 1 to 5. Cells may be prepared from fresh liver, for example, by the well known method of collagenase perfusion (A. P. Li et al., J. Tissue Culture Meth. 14, 139-146 (1992)), and frozen, for example, by the freezing method using a well known program freezer (L. J. Loretz et al., Xenobiotica 19(5), 489-498 (1989)).

Cryopreserved hepatocytes were rapidly thawed at 37° C., and then washed twice with L-15 medium containing 10% fetal bovine serum, followed by suspending in Landford's medium supplemented with 10% fetal bovine serum (R. Lanford et al., In Vitro Cellular & Developmental Biology 25(2), 174-182 (1989)). The cells in the suspension were seeded in a 12-well culture plate coated with collagen at the density of 6×10⁵ cells/well, and the plate was incubated one day and night in the CO₂ incubator. Subsequently, the culture medium containing 10% fetal bovine serum was removed, 1 ml of fresh serum-free Lanford's medium was added to each well, and the cells were maintained in the CO₂ incubator. The inside of the CO₂ incubator used in the present invention was maintained at 37° C. in an atmosphere of 5% carbon oxide saturated with vapor.

### EXAMPLE 2

### Activity Measurement of Drug Metabolizing Enzymes

Culture medium was removed from the culture plate in which the cells were maintained, and given enzyme reactions were added to measure the enzyme activities shown below.

(1) Measurement of Testosterone Hydroxylation Activity

Lanford's medium (1 mL) containing 250 μmol/L of testosterone was added to each well after the culture medium was removed, and the plate was incubated for two hours in the CO₂ incubator to allow reaction. All the volume of each reaction was recovered and stored at −80° C. until measurement. The cells were washed twice with Lanford's medium, and maintained in the CO₂ incubator again or directly used as a sample for another test. The stored reactions were thawed, and accurately 0.4 mL of each reaction was removed and mixed with 0.1 mL of internal standard solution containing 20 μmol/L of 11α-hydoroxyprogesterone. The reactant was extracted from this mixture with 2 mL of ethyl acetate. Whole volume of the ethyl acetate extract phase was collected in another vessel. Ethyl acetate was evaporated and the residual reactant was redissolved in 0.4 mL of the mobile phase consisting of MeOH:CH₃CN:H₂O=40:5:55. From this solution, 50 μl was subjected to liquid chromatography. Testosterone hydroxylation activity was measured based on the resulting products of 6β-hydroxylated testosterone.

(2) Measurement of Ethoxyresorfin Dealkylation Activity

After the culture medium was completely removed from the culture plate, Lanford's medium (1 mL) containing 8 μmol/L of 7-ethoxyresorfin and 10 μmol/L of dicumarol was added to each well, and the plate was incubated for 30 minutes in the CO₂ incubator to allow reaction. Whole volume of each reaction was recovered and stored at −80° C. until measurement. The cells were washed twice with Lanford's medium, and maintained in the CO₂ incubator again or directly used as a sample for another test.

The stored reactions were thawed, and accurately 75 μL of each reaction was removed and mixed with 0.1 mol/L of acetate-sodium acetate buffer (pH 4.4, 25 μL) containing 15 Fishman units of β-glucuronidase [EC 3.2.1.31] and 120 Roy units of sulfatase [EC 3.1.6.1], followed by conjugation reaction for two hours at 37° C. Ethanol (200 μL) was added to the reaction and subjected to centrifugation. The supernatant (200 μL) was transferred to a 96-well microtiter plate. Microplate reader (Labsystems, Fluoroscan Ascent) was used to measure the fluorescence intensity at the wavelength of 590 nm when excited with excitation light at the wavelength of 544 nm. The resorfin production figured out from the result was used to measure the ethoxyresorfin dealkylation activity.

(3) Measurement of Ethoxycoumarin Dealkylation and Coumarin Conjugation Activities

Lanford's medium (1 mL) containing 75 μmol/L of 7-ethoxycoumarin was added to each well after the culture medium was removed, and the plate was incubated for two hours in the CO₂ incubator to allow reaction. Whole volume of each reaction was recovered and stored at −80° C. until measurement. The cells were washed twice with Lanford's medium, and maintained in the CO₂ incubator again or directly used as a sample for another test. The stored reactions were thawed, and 50 μl of the reaction was analyzed by liquid chromatography, ethoxycoumarin dealkylation activity and glucuronate and sulfate conjugation activities were comprehensively estimated based on the production of 7-hydroxycoumarin, 7-hydroxycoumarin glucuronide and 7-hydroxycoumarin sulfate. Herein, 7-ethoxycoumarin is converted to 7-hydroxycoumarin after dealkylation, and then conjugated by glucuronate and sulfate.

### EXAMPLE 3

### CYP1A Expression in the Primary Hepatocytes

Human hepatocytes maintained on the 12-well culture plate (HH-110 and HH-118) were exposed to 3-methylcholanthrene or benz[a]anthracene, and the cells collected from four wells for each sample were dissolved in 300 μl of sample buffer containing SDS and heat denatured. 10 μl of the sample was electrophoresed on polyacrylamide gel, and blotted onto PVDF transfer membrane. The transfer membrane was allowed to react with anti-human CYP1A1/2 monoclonal antibody (goat IgG) (Daiichi Pure Chemicals Co., Ltd.) for one hour at room temperature, and protein that had developed color by peroxidase was detected using 4-chloronaphtol method.

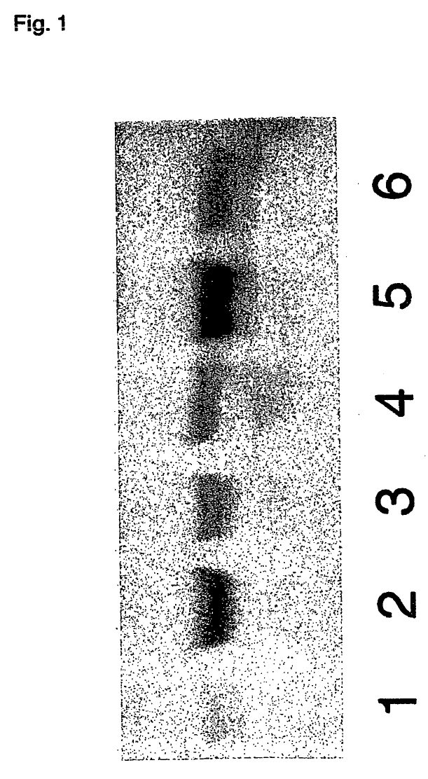

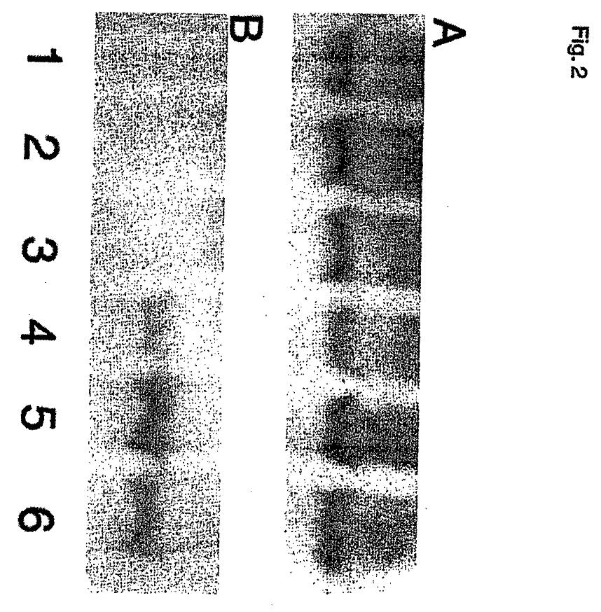

The results are shown in FIG. 1.

The primary human hepatocytes derived from two different donors, HH-118 (Lanes 1, 2 and 3) and HH-110 (Lanes 4, 5 and 6), were loaded without any chemicals (controls) (Lanes 1 and 4), with 3-methylcholanthrene (2 μmol/L) (Lanes 2 and 5) and benz[a]anthracene (5 μmol/L) (Lanes 3 and 6) for one day and night, and served as the cell samples. As a result, both HH-110 and HH-118 exhibited increased CYP1A1/2 expression by 3-methylcholanthrene or benz[a]anthracene.

### EXAMPLE 4

### CYP3A Expression in the Primary Hepatocytes

Human hepatocytes maintained on the 12-well culture plate (HH-110 and HH-118) were loaded to rifampicin or phenobarbital for three days, and the cells collected from four wells for each sample were dissolved in 300 μl of sample buffer containing SDS and heat denatured. 10 μl of the sample was electrophoresed on polyacrylamide gel, and blotted onto PVDF transfer membrane. The transfer membrane was allowed to react with anti-human CYP3A4 monoclonal antibody (goat IgG) (GENTEST) or anti-human CYP3A5 monoclonal antibody (goat IgG) (GENTEST) for one hour at room temperature, followed by further reaction with alkaline phosphatase labelled anti-goat IgG rabbit serum (GENTEST) for one hour at temperature. Protein that had developed color by alkaline phosphatase was detected, using the BCIP/NBT method.

The results are shown in FIG. 2

The primary human hepatocytes derived from two different donors, HH-118 (Lanes 1, 2 and 3) and HH-110 (Lanes 4, 5 and 6), were loaded for three days without any chemicals (controls) (Lanes 1 and 4), with rifampicin (10 μmol/L) (Lanes 2 and 5) and phenobarbital (1 mmol/L) (Lanes 3 and 6), and served as the cell samples. For primary antibody, anti-human CYP3A4 goat IgG antibody (FIG. 2A), or anti-human CYP3A5 goat IgG antibody (FIG. 2B) was used.

As a result, both HH-118 and HH-110 exhibited increased CYP3A4 expression by rifampicin, but effect of phenobarbital was not marked. Also, in HH-110 CYP3A5 was confirmed to be expressed, and its expression level was increased by rifampicin.

### EXAMPLE 5

### Recovery of Total RNA From Primary Hepatocytes

RNeasy Mini Kit (QIAGEN) was used to recover total RNA from primary hepatocytes (HH-018). Specifically, culture medium or enzyme reaction solution was removed from the culture plate in which the cells were maintained, and 0.35 mL of cell lysis buffer (Buffer RLT supplemented with 1% 2-mercaptoethanol) in the above kit was added to each well. The whole cell lysate thus obtained was used for total RNA purification, according to the instruction attached to the kit. Purified RNA eluted through the Mini column with 30 μL of RNase-free purified water was assayed according to a well-known method, to determine its density by measuring the absorbance at the wavelength of 260 nm and to determine the purity by calculating the ratio of the absorbance at 260 nm to 280 nm. Generally, 3 to 10 μg of total RNA was obtained from the cells in each well, and the value of A260/A280 that indicates the purity of RNA was 1.9 or more. Total RNA sample thus obtained includes ribosomal RNA, transfer RNA and messenger RNA (mRNA).

### EXAMPLE 6

### Analysis of Cytochrome P450 Gene Expression

Reverse transcription was carried out using Thermoscript RT-PCR kit (GIBCO BRL) and, as template, 500 ng of the total RNA for each sample obtained in Example 5, according to the attached instruction. To investigate expression of each cytochrome P450, mRNA levels were analyzed using cDNA obtained by the reverse transcription as template, by a well known method PCR using DNA primers specific for each gene. Expression level of β-actin, which is nearly constant and may serve as a reference of mRNA, was also analyzed. The primers used for PCR were prepared from the particular base sequences available from the Gene Bank database. Gene Bank accession numbers for those sequences are X00351 for β-actin, K03191 for CYP1A1, M55053 for CYP1A2, X13897 for CYP2A6, M29874 for CYP2B6, M17397 for CYP2C8, M61857 for CYP2C9, M61854 for CYP2C19, X08006 for CYP2D6, J02625 for CYP2E1, J04449 for CYP3A4, J04813 for CYP3A5, and D00408 for CYP3A7. These sequences of individual primers are shown under SEQ ID Nos. 1-16 in the Sequence Listing. Among them, CYP1A1 and CYP1A2 are simultaneously amplified with a same set of primers (SEQ ID Nos. 3 and 4)(CYP1A1/2), and CYP2C8, CYP2C9 and CY2C19 are simultaneously amplified with another set of primers (SEQ ID Nos. 9 and 10)(CYP2C8/9/19). Likewise, CYP3A4, CYP3A5 and CYP3A7 are simultaneously amplified with another set of primers (SEQ ID Nos. 15 and 16)(CYP3A4/5/7). Annealing temperature in the PCR was 60° C. for β-actin, CYP2A6, CYP2B6 and CYP2C8/9/19, 63° C. for CYPA1/2 and CYP2D6, 57° C. for CYPA2E1 and CYP3A4/5/7. PCR was performed at the cycle number of 18-30.

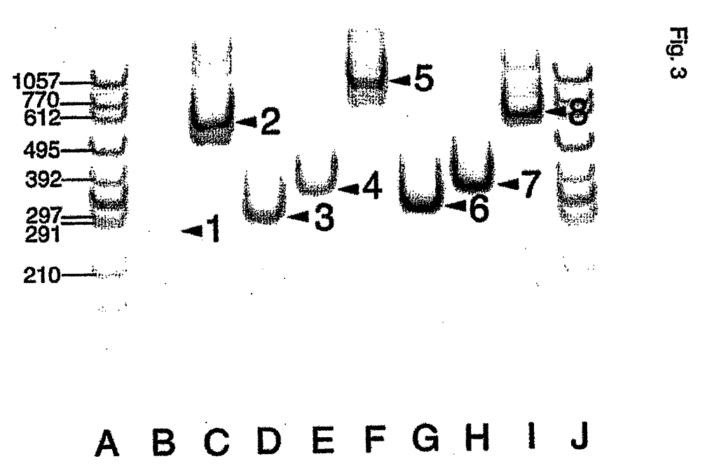

FIG. 3 shows the results of polacrylamide gel electrophoresis of the RT-PCR products amplified using total RNA extracted from primary human hepatocytes HH-018 as templates, along with DNA molecular weight standards (φX174/Hinc II: a sample of plasmid φX174 cleaved completely with Hinc II).

Molecular weight marker φX174/Hinc II was applied to lanes A and J for electrophoresis, and the PCR products amplified with the primers specific for the respective genes as shown below were loaded on the other lanes; B: β-actin, C: CYP1A1/2, D: CYP2A6, E: CYP2B6, F: CYP2C8/9/19, G: CYP2D6, H: CYP2E1, I: CYP3A4/5/7. The chain length (number of base pairs) of each molecular weight marker is indicated on the left side of Lane A.

For electrophoretic mobility of the individual genes obtained from the primary hepatocyte cDNA, predicted sizes (275 bp for β-actin: amplified product 1, 663 bp for CYP1A1/2: amplified product 2, 306 bp for CYP2A6: amplified product 3, 377 bp for CYP2B6: amplified product 4, 840 bp for CYP2C8/9/19: amplified product 5, 333 bp for CYP2D6: amplified product 6, 366 bp for CYP2E1: amplified product 7, and 618 bp for CYP3A4/5/7: amplified product 8) were almost identical to the electrophoresis relative to the electrophoresis of the respective fragments of the DNA molecular standard.

### EXAMPLE 7

### Subtype Analysis of Cytochrome P450

Among the PCR amplified products of CYPs, CYP1A1/2, CYP2C8/9/19 and CYP3A4/5/7 can be classified into subtypes, depending on whether those products are cleaved with particular restriction enzymes commercially available. The restriction enzymes and restriction sites used were Nae I: GCCGGC, Pst I: CTGCAG, Hpa I: GTTAAC, Bgl II: AGATCT, Pvu II: CAGCTG, Bam HI: GGATCC, Nsp V: TTCGAA, and Hind III: AAGCTT. Actual procedure that was carried out using the PCR-amplified products obtained from the plasmids expressing respective CYP genes and the results obtained are as follows:

(1) Separation of PCR Fragments of CYP1A1 and CYP1A2

It was presumed that Nae I would cleave CYP1A1 into two fragments of 134 and 529 bp in length but not CYP1A2. Pst I was presumed to cleave CYP1A2 into three fragments of 42, 264 and 356 bp in length but not CYP1A1.

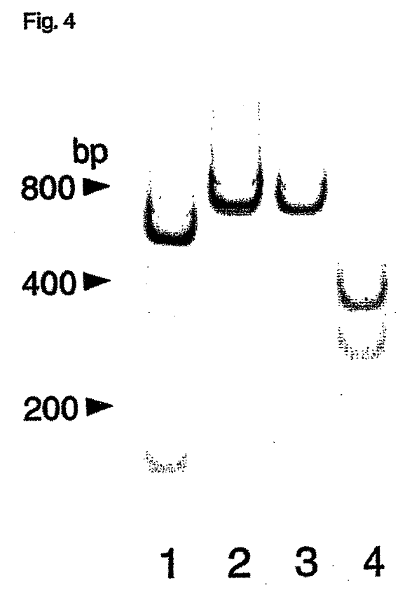

The results are shown in FIG. 4. PCR-amplified products from the plasmid expressing the CYP1A1 gene (Lanes 1 and 2) and the plasmid expressing the CYP1A2 gene (Lanes 3 and 4) were digested with Nae I (Lanes 1 and 3) and Pst I (Lanes 2 and 4) and then subjected to polyacrylamide gel electrophresis. As expected, Nae I cleaved CYP1A1 into two fragments of 134 and 529 bp in length but not CYP1A2, and Pst I cleaved CYP1A2 into three fragments of 42, 264 and 356 bp in length but not CYP1A1.

(2) Separation of PCR Fragments of CYP2C8, CYP2C9 and CYP2C19

It was presumed that Hpa I would cleave CYP2C8 into two fragments of 316 and 524 bp in length but not 2C9 and 2C19. Bgl II was presumed to cleave CYP2C9 into two fragments of 316 and 524 bp in length but not 2C8 and 2C19. Pvu II was presumed to cleave CYP2C19 into two fragments of 420 bp in length but not 2C8 and 2C9.

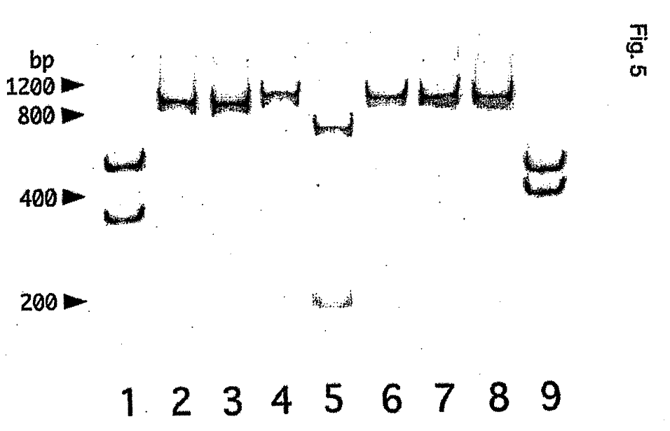

The results are shown in FIG. 5.

PCR-amplified products from the plasmid expressing the CYP2C8 gene (Lanes 1, 2 and 3), the plasmid expressing the CYP2C9 gene (Lanes 4, 5 and 6), and the plasmid expressing the CYP2C19 gene (Lanes 7, 8 and 9) were digested with Hpa I (Lanes 1, 4 and 7), Bgl II (Lanes 2, 5 and 8), and Pvu II (Lanes 3, 6 and 9), respectively and then subjected to polyacrylamide gel electrophoresis. As expected, Hpa I cleaved CYP2C8 into two fragments in length but not 2C9 and 2C19. Bgl II cleaved CYP2C9 into two fragments but not 2C8 and 2C19. Pvu II cleaved CYP2C19 into two fragments but not 2C8 and 2C9.

The separation of the two Pvu II fragments of CY2C19 expected to have the same size may be due to difference in composition of these fragments. When these fragments were electrophoresed on agarose gel, such separation was not observed.

(3) Separation of PCR Fragments of CYP3A4, CYP3A5 and CYP3A7

It was presumed that Bam HI would cleave CYP3A4 into two fragments of 285 and 333 bp in length but not 3A5 and 3A7. Nsp V was presumed to cleave CYP3A5 into two fragments of 144 and 477 bp in length but not 3A4 and 3A7. Hind III was presumed to cleave CYP3A4 and CYP3A7 into two fragments of 262 and 356 bp in length but not 3A5.

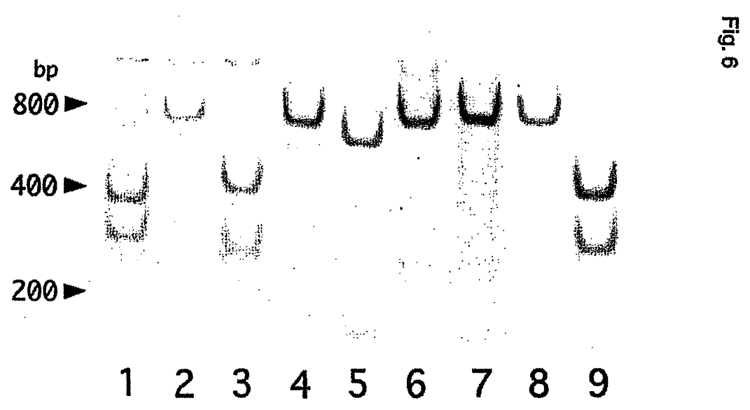

The results are shown in FIG. 6.

PCR-amplified products from the plasmid expressing the CYP3A4 gene (Lanes 1, 2 and 3), the plasmid expressing the CYP3A5 gene (Lanes 4, 5 and 6), and the plasmid expressing the CYP3A7 gene (Lanes 7, 8 and 9) were digested with Bam HI (Lanes 1, 4 and 7), Nsp V (Lanes 2, 5 and 8), and Hind III (Lanes 3, 6 and 9), and then subjected to polyacrylamide gel electrophoresis. As expected, Bam HI cleaved CYP3A4 into two fragments but not 3A5 and 3A7. Nsp V cleaved CYP3A5 into two fragments but not 3A4 and 3A7. Hind III cleaved CYP3A4 and CYP3A7 into two fragments but not 3A5.

### EXAMPLE 8

### Effect of the Maintenance Method on Induction of Enzyme Activity

As described above, cryopreserved human hepatocytes (HH-110) were thawed and seeded in a 12-well culture plate coated with collagen. The cells were maintained in Lanford's medium one day and night, culture medium was removed, and 1) 1 ml of fresh serum-free Lanford's medium containing 10 μmol/L of rifampicin, a well known CYP3A-inducing agent, was added to each well and maintained in the CO₂ incubator for three days, 2) fresh serum-free Lanford's medium containing 10 μmol/L of rifampicin was added to the well and maintained in the CO₂ incubator for three days, replacing with fresh medium of the same composition every 24 hours, or 3) 1 ml of fresh serum-free Lanford's medium was added to each well and maintained in the CO₂ incubator for two days, then the medium was replaced with fresh serum-free Lanford's medium containing 10 μmol/L of rifampicin, and the culture was maintained in the CO₂ incubator for further three days, replacing with fresh medium having the same composition every 24 hours. Subsequently, testosterone hydroxylation activity was measured for each cell. As a negative control of enzyme induction for each condition, the cells maintained in the rifampicin-free medium were used.

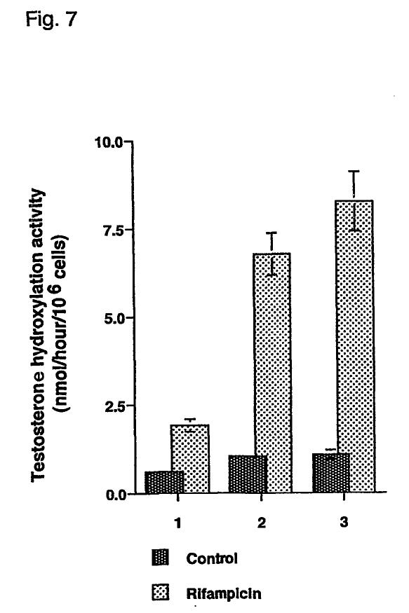

The results are shown in FIG. 7.

Testosterone hydroxylation activity was measured separately in three samples for each condition, using primary human hepatocytes (HH-110) maintained for three days in the medium replaced with the medium containing 10 μmol/L of rifampicin (Lane 1), maintained in the fresh medium replaced with one containing 10 μmol/L of rifampicin for three days, replacing with fresh medium having the same composition every 24 hours (Lane 2), and maintained in the fresh medium for two days, and further for three days in the medium replaced with fresh medium containing 10 μmol/L of rifampicin, replacing it with another fresh medium having the same composition every 24 hours (Lane 3). Mean values are shown in the graph, and standard deviations are indicated by error lines.

Replacement with fresh medium at 24-hour intervals led to a three-fold or more increase in the testosterone hydroxylation activity after induction, compared to the case where the medium was not replaced. In this instance, higher activity was also obtained in the control cells that had medium replacement than those without replacement. Slightly higher testosterone hydroxylation activity after the induction was obtained when three-day pre-culturing was provided between adhesion of the cells on the plate and rifampicin treatment, compared to the case where no pre-culturing was provided.

### EXAMPLE 9

### Effect of the Components of the Culture Medium on the Induction of Enzyme Activity

Among the components of Landford's medium used for culture, the effects of prolactin, cholera toxin, liver cell growth factor (LCGF) and hydrocortisone on the enzyme activities involved in xenobiotic metabolism were investigated. That is, as Landford's medium that was used throughout the entire period of seeding, pre-culturing, maintenance of the cells and assaying of the compound, 1) prolactin-free medium, 2) cholera toxin-free medium, 3) liver cell growth factor (LCGF)-free medium, and 4) hydroxycortisone-free medium were used. Testosterone hydroxylation activity of each culture after induction by rifampicin was measured. HH-110 cells were used.

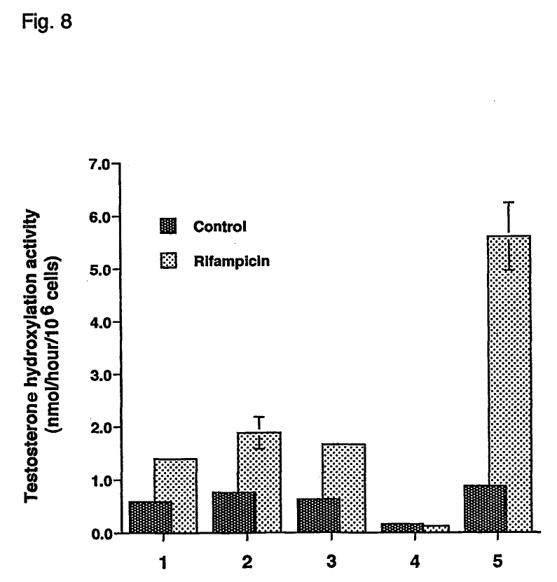

The results are shown in FIG. 8.

Three samples of primary human hepatocytes (HH-110) for each condition were independently assayed for their testosterone hydroxylation activity, using prolactin-free medium (Lane 1), cholera toxin-free medium (Lane 2), liver cell growth factor (LCGF)-free medium (Lane 3), hydrocortisone-free medium (Lane 4) and conventional Landford's medium (Lane 5), and their mean values are shown, and standard deviations are indicated by error lines.

Prolactin, cholera toxin and liver cell growth factor (LCGF) are all enhancing factors for induction of testosterone hydroxylation activity. Hydrocortisone was essential for maintenance of testosterone hydroxylation activity per se.

Then, the effect of Landford's medium containing 10% fetal bovine serum was monitored during one day and night, between seeding and adhesion of the cells. HH-110 cells were used.

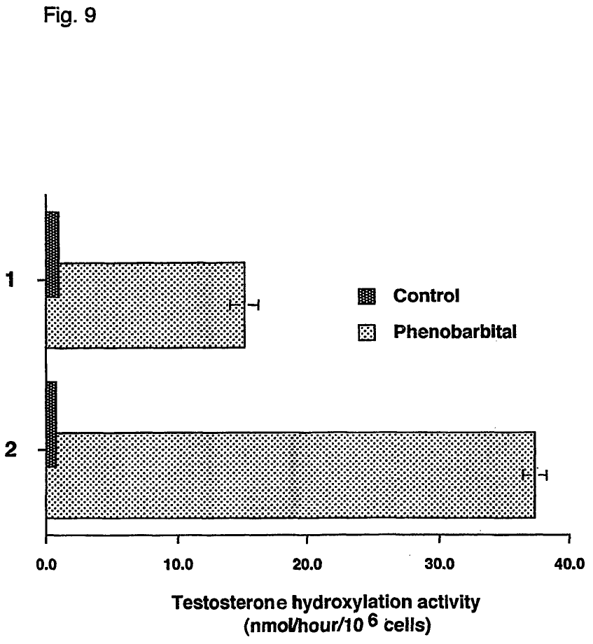

The results are shown in FIG. 9.

Primary human hepatocytes (HH-110) were maintained in serum-free Landford's medium (Lane 1) or Landford's medium containing 10% fetal bovine serum (Lane 2) one day and night. Subsequently, the culture medium was replaced with medium containing 10 μmol/L of rifampicin and maintained for three days, with the medium replaced with fresh medium having the same composition at 24-hour intervals. Testosterone hydroxylation activity of these cells was measured separately in three samples for each condition, and the mean values are shown. The standard deviations are indicated by error lines.

Use of serum-containing medium for one day and night after seeding of the cells improved the viability of the cells, and the testosterone hydroxylation activity after the phenobarbital induction increased twice or more, compared to the case where serum-free medium was used.

From the results described above, the inventors found that, to maintain cryopreserved human hepatocytes while retaining high the functions involved in xenobiotic metabolism in the liver, it is desirable 1) to adhere the cells to a 12-well culture plate coated with collagen by incubating for one day and night in Lanford's medium containing 10% fetal bovine serum after the cells are thawed; 2) to replace with fresh serum-free Lanford's medium and maintain the cells for three days in the CO₂ incubator without further replacement of the medium; 3) to add fresh serum-free Lanford's medium containing a test compound; and 4) to maintain the cells in the CO₂ incubator, with replacing the medium with fresh medium having the same composition at 24 hour intervals. Moreover, the inventors found that prolactin, cholera toxin, liver cell growth factor (LCGF) and hydrocortisone are important for testosterone hydroxylation activity and its induction.

### EXAMPLE 10

### Changes in Testosterone Hydroxylation Activity After Induction by Chemical Agents With Time

The cells maintained under the conditions considered optimal in Examples 8 and 9 had been loaded with 10 μmol/L of rifampicin or 1 mmol/L of phenobarbital for four days, and the changes in testosterone hydroxylation activity was investigated. HH-110 cells were used.

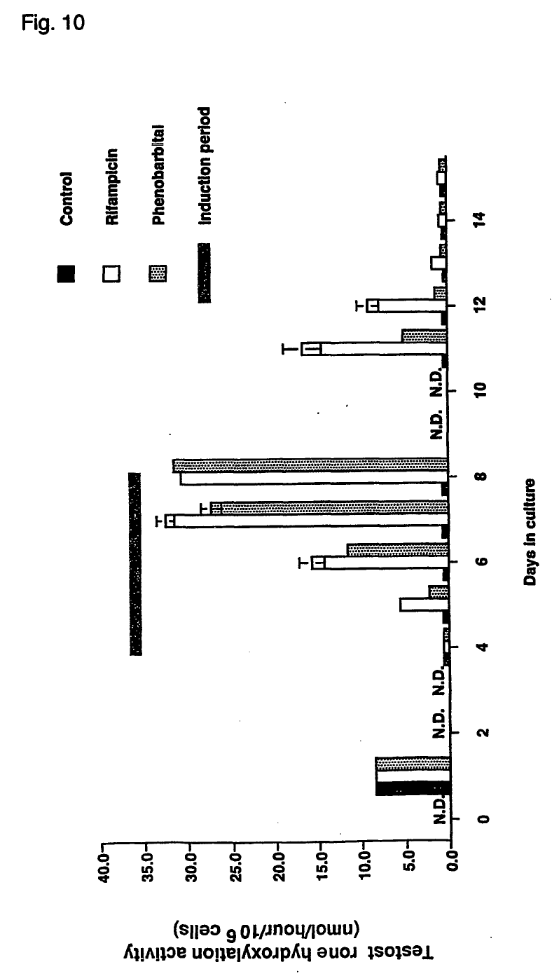

The results are shown in FIG. 10.

Primary human hepatocytes (HH-110) had been loaded with 10 μmol/L of rifampicin or 1 mmol/L of phenobarbital for four days between days 4th to 8th post seeding (indicated by a crossbar in the figure), and these cells were maintained further for one week again in the conventional Lanford's medium. During this period, measurement of testosterone hydroxylation activity was conducted at 11 times to investigate the changes in the activity. Three samples per condition were measured independently, and the mean values are shown. The standard deviations are indicated by error bar.

The days without measuring the activity are indicated by N.D.

Maximum testosterone hydroxylation activity was observed at day 3 in the cells to which 10 μmol/L of rifampicin was added, and at day 4 in the cells to which 1 mmol/L of phenobarbital is added. In either case, the cells at days 3 and 4 after addition of the compound had almost equal levels of activity, and they are considered to reach a maximum. In addition, when the addition of the compounds was stopped, the testosterone hydroxylation activity was rapidly decreased and after five days, returned to their original state prior to the addition of the compounds.

### Example 11

### Individual Difference in the Testosterone Hydroxylation Activity and its Induction

The cells prepared from five different donors (HH-018, HH-022, HH-029, HH-110 and HH-118) were loaded with 10 μmol/L of rifampicin or 1 mmol/L of phenobarbital for four days under the conditions considered optimal in Examples 8 and 9. Subsequently, measurements of testosterone hydroxylation activity and mRNA analysis were conducted.

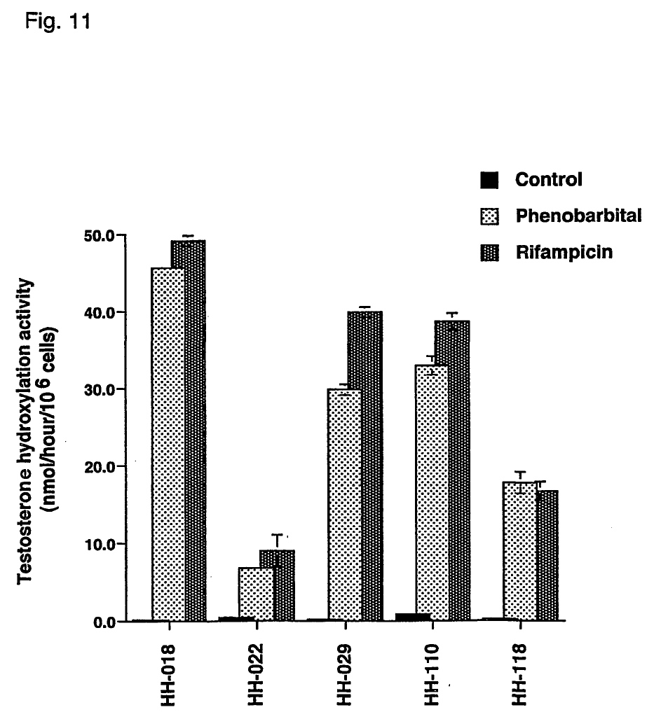

The results of the measurement of the testosterone hydroxylation activity are shown in FIG. 11.

Three samples per condition were measured independently, and the mean values are shown. The standard deviations are indicated by error bar.

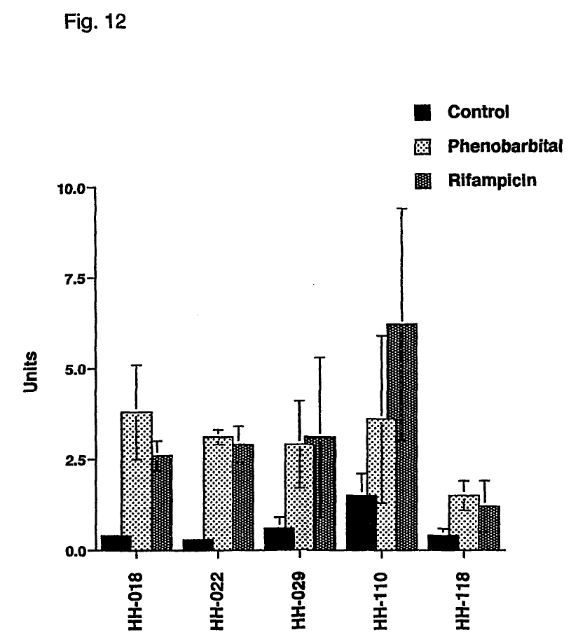

Quantification of CYP3A mRNA is shown in FIG. 12. PCR was performed with 27 cycles for CYP3A, and 18 cycles for β-actin, and the ratio of the products obtained (CYP3A (ng)/β-actin (ng)) was defined as unit for comparison.

Three samples per condition were measured independently, and the mean values are shown. The standard deviations are indicated by error bar.

Increases of testosterone hydroxylation activity and CYP 3A mRNA mediated by rifampicin and phenobarbital were observed in all the cells.

Although HH-022 showed a reduced activity, as revealed by the mRNA analysis, the rate of induction of CYP 3A gene, which was normalized using the levels of β-actin, did not much differ. Therefore, this is likely due to the reduced cell adhesion rate. Although HH-118 exhibited somewhat lower levels of enzyme activity and mRNA induction than other cells, this is likely due to individual difference in inducing property.

Thus, by using this method, even if cells from different donors are used, it is possible to be examined by same method, and the effects of test compounds on the cells from plural different donors can be determined simultaneously under the same conditions. Therefore, the individual difference of the effect of a compound on testosterone hydroxylation activity in human hepatocytes can be examined.

### EXAMPLE 12

### Changes in Ethoxyresorfin Dealkylation Activity After Induction by Chemical Agents With Time

The cells maintained under the conditions considered optimal in Examples 8 and 9 were continued to be added with 1 μmol/L of 3-methylcholanthrene for three days, and the changes in ethoxyresorfin dealkylation activity was investigated. HH-110 cells were used.

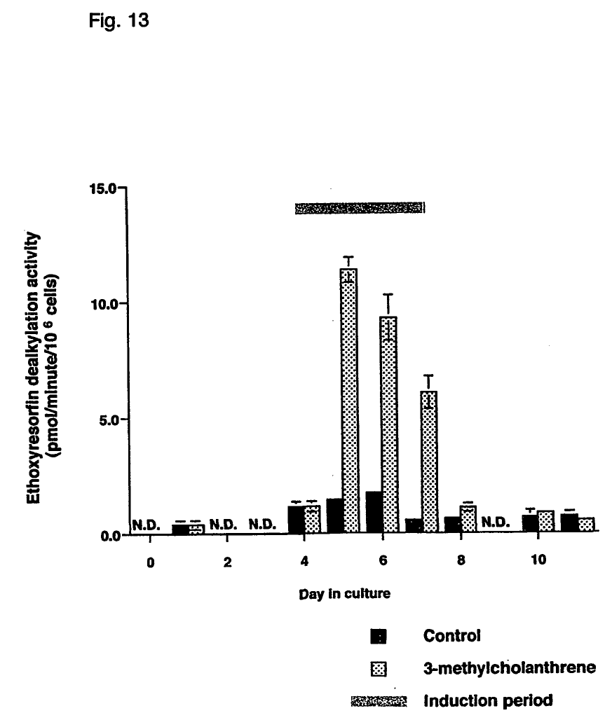

The results are shown in FIG. 13.

Primary human hepatocytes (HH-110) were continued to be loaded with 1 μmol/L of 3-methylcholanthrene for three days between days 4th to 7th post seeding (indicated by a crossbar in the figure), and these cells had been maintained further for five days again in the conventional Lanford's medium. During this period, measurement of ethoxyresorfin dealkylation activity was conducted at 8 times to investigate the changes in the activity. Three samples per condition were measured independently, and the mean values are shown. The standard deviations are indicated by error bar.

The days without measuring the activity are indicated by N.D.

Maximum activity was achieved one day after 3-methylcholanthrene was added, and the activity gradually decreased even if the compound was continued to be added. When the addition of the compounds was stopped, the ethoxyresorfin dealkylation activity was rapidly decreased and returned to their original state prior to the addition of the compounds in a day.

### EXAMPLE 13

### Individual Difference in the Ethoxyresorfin Dealkylation Activity and its Induction

The cells prepared from five different donors (HH-018, HH-022, HH-029, HH-110 and HH-118) were loaded with 2 μmol/L of benz[a]anthracene or 1 μmol/L of 3-methylcholanthrene for two days under the conditions considered optimal in Examples 8 and 9. Subsequently, measurements of ethoxyresorfin dealkylation activity and mRNA analysis were conducted. mRNA analysis of HH-029 was conducted only on the cells loaded with 1 μmol/L of 3-methylcholanthrene.

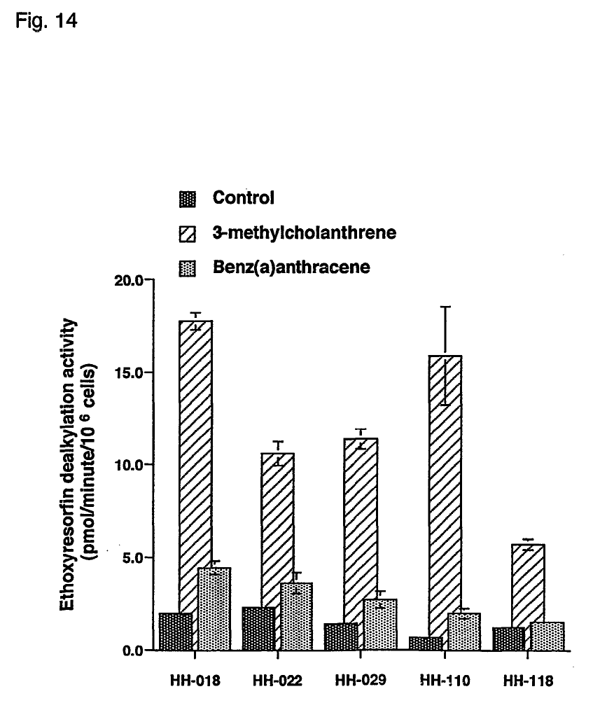

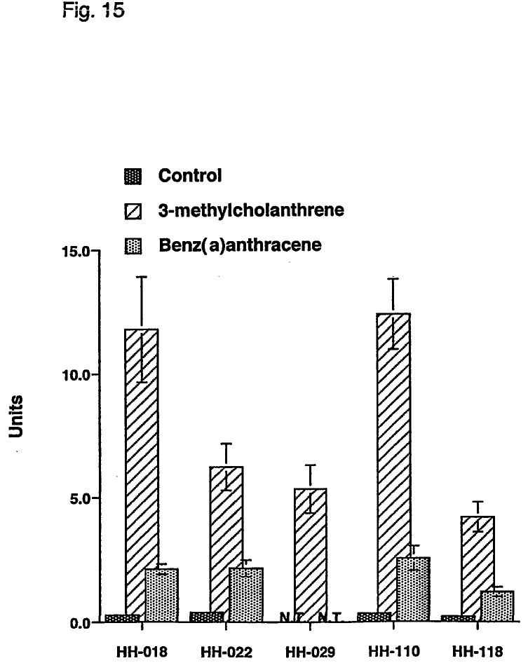

The results of the measurement of the ethoxyresorfin dealkylation activity are shown in FIG. 14.

Three samples per condition were measured independently, and the mean values are shown. The standard deviations are indicated by error bar.

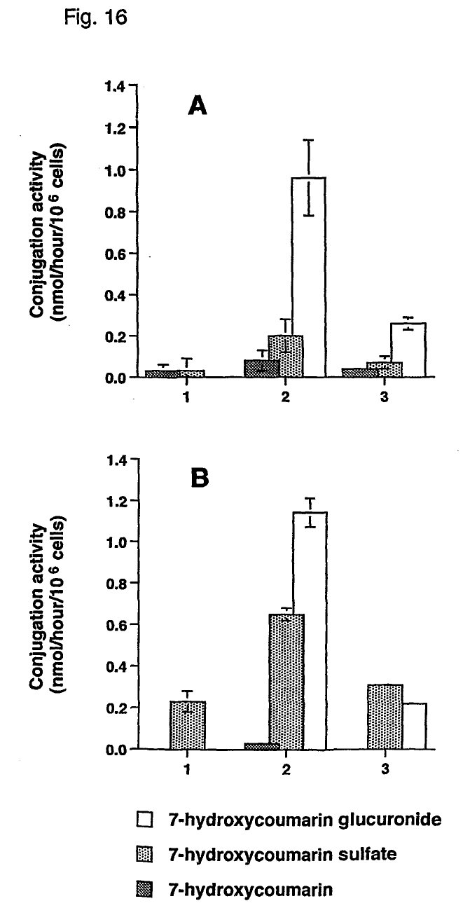

Quantification of CYP1A mRNA is shown in FIG. 15. PCR was performed with 23 cycles for CYP1A, and 19 cycles for β-actin, and the ratio of the products obtained (CYP1A (ng)/β-actin (ng)) was defined as unit for comparison. Three samples per condition were measured independently, and the mean values are shown. The standard deviations are indicated by error bar.

The results from measurement of enzyme activity and mRNA analysis were well agreed, which showed that any cells can be used to estimate induction.

Thus, by using this method, even if cells from different donors are used, it is possible to be examined by same method, and the effects of test compounds on the cells from plural different donors can be determined simultaneously under the same conditions. Therefore, the individual difference of the effect of a compound on ethoxyresorfin dealkylation activity in human hepatocytes can be examined.

### EXAMPLE 14

### Changes in Ethoxycoumarin Dealkylation and Conjugation Activities by Benz[a]anthracene and 3-methylcholanthrene

The cells maintained under the conditions considered optimal in Examples 8 and 9 were continued to be added with 5 μmol/L of benz[a]anthracene or 2 μmol/L of 3-methylcholanthrene one day and night, and then the changes in ethoxycoumarin dealkylation activity was investigated. HH-110 and HH-118 cells were used.

The results using HH-110 are shown in FIG. 16A, and those using HH-118 are shown in FIG. 16B.

Three samples per each condition i.e., control (Lane 1), adding of 3-methylcholanthrene (Lane 2) and benz[a]anthracene (Lane 3) which were measured independently, and the mean values are shown. The standard deviations are indicated by error bar.

In both cells, increases of production of 7-hydroxycoumarin, 7-hydroxycoumarin glucronide, and 7-hydroxycoumarin sulfate by benz[a]anthracene and 3-methylcholanthrene were observed. Thus, even if the cells from different donors are used, it is shown to allow determination of conjugation activity in human hepatocytes by same method. Since production ratio of conjugate differs depending on the donors, it is also possible to investigate individual difference in conjugation activity.

### EXAMPLE 15

### Concentration-dependence of Induction of CYP3A by Various CYP3A Inducing Agents

Rifampicin, clotrimazole, carbamazepine, phenobarbital and dexamethasone, all known as CYP3A inducing agents, were added to primary human hepatocytes (HH-110) at different concentrations to observe increase of testosterone hydroxylation activity and CYP3A mRNA level.

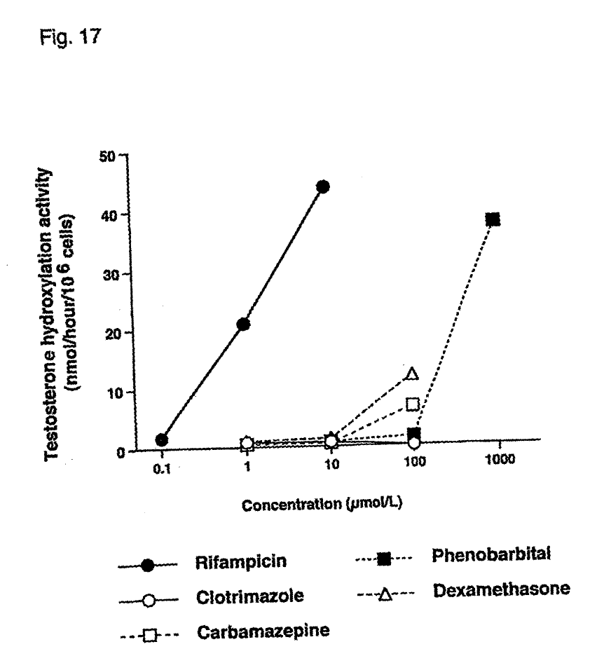

Measurements of testosterone hydroxylation activity are shown in FIG. 17.

Three samples per condition were measured independently, and the mean values are shown. The standard deviations are indicated by error bar.

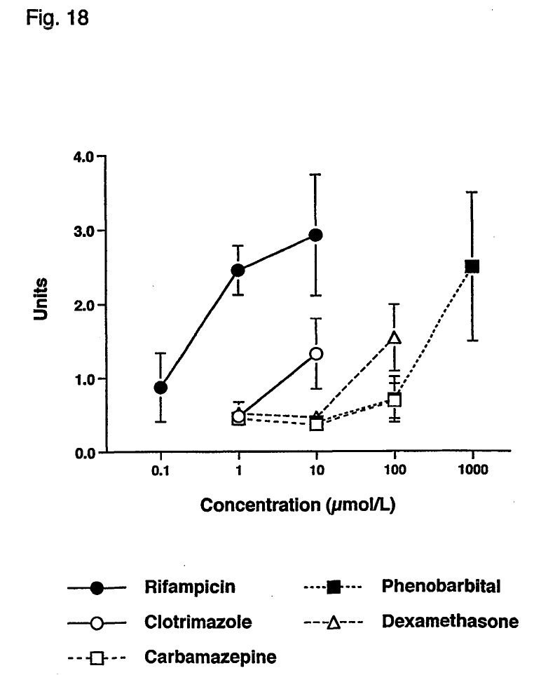

Quantification of CYP3A mRNA is shown in FIG. 18. PCR was performed with 27 cycles for CYP3A, and 18 cycles for β-actin, and the ratio of the products obtained (CYP3A (ng)/β-actin (ng)) was defined as unit for comparison.

Three samples per condition were measured independently, and the mean values are shown. The standard deviations are indicated by error bar.

In the clotrimazole-added cells, increase of the mRNA level was observed but increase of the testosterone hydroxylation activity was not observed. It is considered that clotrimazole inhibits testosterone hydroxylation activity by enzyme inhibitory effect or toxicity. With regard to other compounds, the results from measurement of enzyme activity and mRNA analysis were well agreed, and concentration-dependent induction was observed.

### EXAMPLE 16

### Concentration-dependence of Induction of CYP1A by 3-methylcholanthrene and Benz[a]anthracene

3-methylcholanthrene and benz[a]anthracene, known as CYP1A inducing agents, were added to primary human hepatocytes (HH-029) at different concentrations to observe increase of ethoxyresorfin dealkylation activity and CYP1A mRNA level.

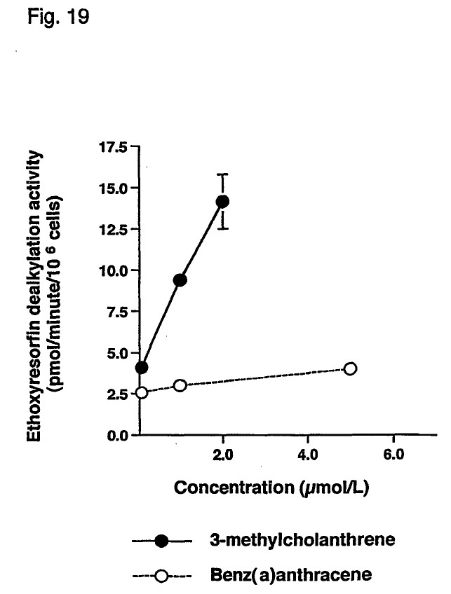

Measurements of ethoxyresorfin dealkylation activity are shown in FIG. 19.

Three samples per condition were measured independently, and the mean values are shown. The standard deviations are indicated by error bar.

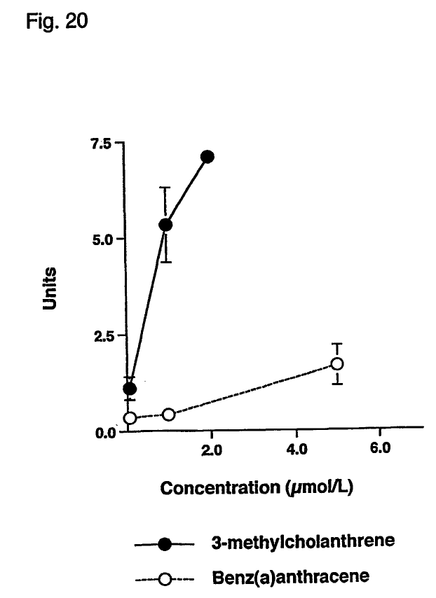

Quantification of CYP1A mRNA is shown in FIG. 20. PCR was performed with 23 cycles for CYP1A, and 19 cycles for β-actin, and the ratio of the products obtained (CYP1A (ng)/β-actin (ng)) was defined as unit for comparison.

Three samples per condition were measured independently, and the mean values are shown. The standard deviations are indicated by error bar.

The results from measurement of enzyme activity and mRNA analysis were well agreed, and concentration-dependent induction was confirmed.

### EXAMPLE 17

### The Testosterone Hydroxylation Activity and its Induction by Using Cryopreserved Primary Human Hepatocytes Purchased From Different Suppliers

The cells prepared from seven different donors that were different from the cells of Example 1 were loaded with 10 μmol/L of rifampicin for 3 days under the condition considered optimal in Example 8 and 9. Subsequently, measurement of testosterone hydroxylation was conducted.

Cryopreserved primary human hepatocytes prepared from seven different donors were purchased from Tissue Transformation Technology (NJ, USA), In Vitro Technologies, Inc. (MD, USA) and XenoTech, LLC (KS, USA). The cells named HH-135 and HH-148 were prepared by Tissue Transformation Technology (NJ, USA), the cells named IVT-077, IVT-088, IVT-100 and IVT-124 were prepared by In Vitro Technologies, Inc. (MD, USA) and the cell named XEN-254 was prepared by XenoTech, LLC (KS, USA). Information on the donors is shown below as hepatocytes donors 6 to 12.

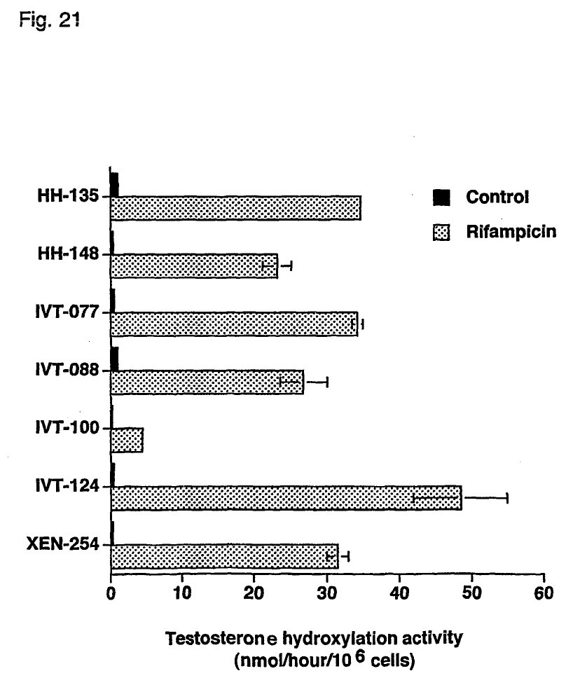

The results of the measurement of the testosterone hydroxylation activity are shown in FIG. 21.

Three samples per condition were measured independently, and the mean values are shown. The standard deviations are indicated by error bar.

Increases of testosterone hydroxylation activity were observed in all the cells.

Thus, by using this method, even if cells purchased from different suppliers were used, it is possible to be examined by same method.

### EXAMPLE 18

### Effect of Hydrocortisone on Induction of Testosterone Hydroxylation Activity

Among the conditions considered optimal in Example 8 and 9, the mediums in which the concentration of hydrocortisone was changed were used. The cells maintained under the condition above were continued to be added with 10 μmol/L of rifampicin for three days, and the measurement of testosterone hydroxylation activity was conducted. HH-135 and IVT-088 cells were used.

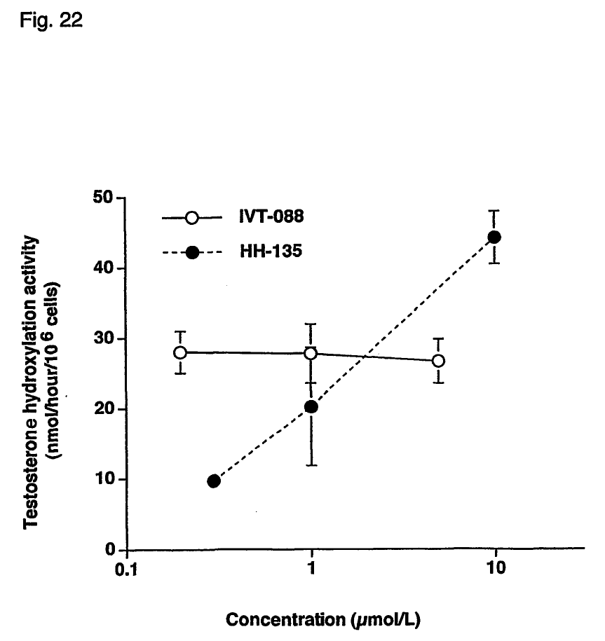

The results are shown in FIG. 22.

Three samples per condition were measured independently, and the mean values are shown. The standard deviations are indicated by error bar.

Although concentration-dependence of induction was different by the cells used, 1 to 10 μmol/L of hydrocortisone was effective on the induction of testosterone hydroxylation activity.

### EXAMPLE 19

### Effect of Glucocorticoid on Induction of Testosterone Hydroxylation Activity

Among the conditions considered optimal in Example 8 and 9, the mediums containing 1 μmol/L of dexamethasone or 1 μmol/L of predonisolone instead of hydrocortisone were used. The cells maintained under the condition above were continued to be added with 10 μmol/L of rifampicin for three days, and the measurement of testosterone hydroxylation activity was conducted. And the results were compared with the result of the condition using mediums containing 1 μmol/L of hydrocortisone. HH-110 cells were used.

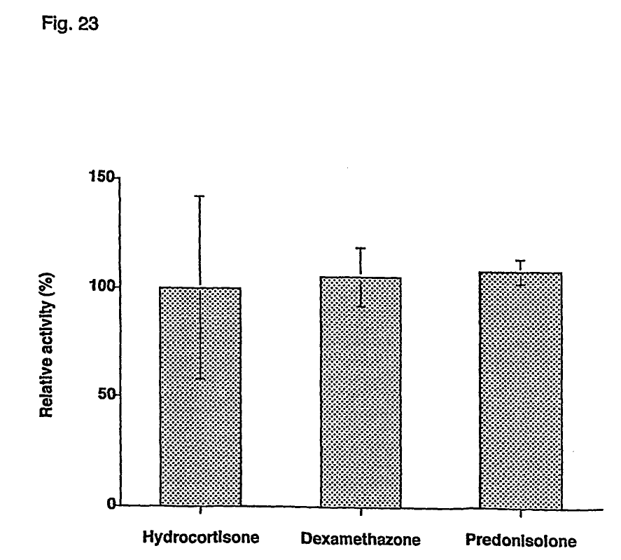

The results are shown in FIG. 23.

The graph shows the relative activities when testosterone hydroxylation activity by using mediums containing 1 μmol/L of hydrocortisone equals 100%.

Three samples per condition were measured independently, and the mean values are shown. The standard deviations are indicated by error bar.

From these results, induction of testosterone hydroxylation activity can be observed by using dexamethasone or predonisolone instead of hydrocortisone.

### EXAMPLE 20

### Effect of Hydroxy Group of Hydrocortisone on Induction of Testosterone Hydroxylation Activity

Among the conditions considered optimal in Example 8 and 9, the mediums containing one of the component selected from the group consisting of 1 μmol/L of 11β,17α-dihydroxyprogesterone, 1 μmol/L of corticosterone and 1 μmol/L of cortexorone, 1 μmol/L of 11β-hydroxyprogesterone and 17α-hydroxyprogesterone instead of 1 μmol/L of hydrocortizone. The cells maintained under the condition above were continued to be added with 10 μmol/L of rifampicin for three days, and the measurement of testosterone hydroxylation activity was conducted. And the results were compared with the result of the condition by using mediums containing 1 μmol/L of hydrocortisone. HH-110 cells were used.

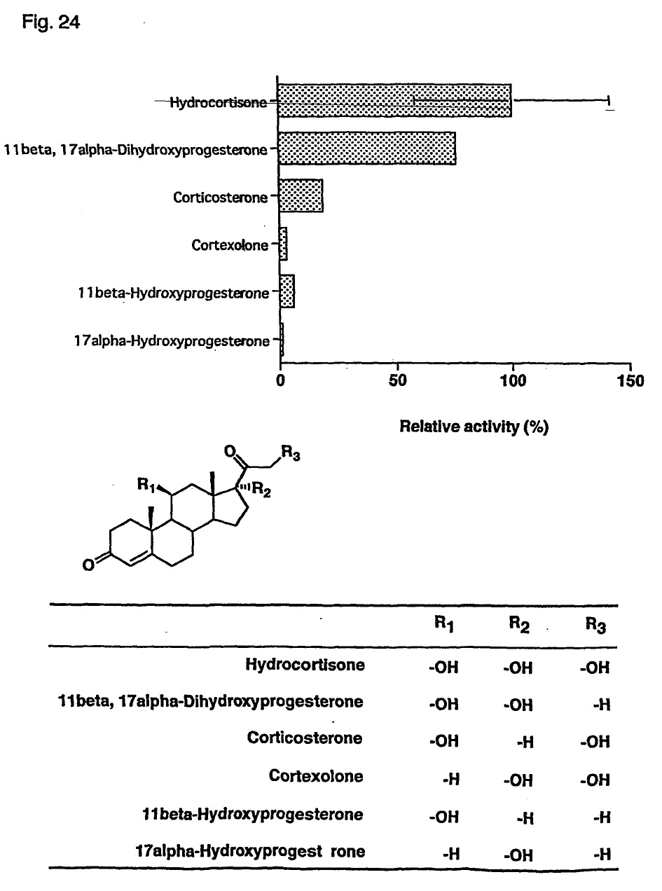

The results and the structures of hydrocortisone analogue are shown in FIG. 24.

The graph shows the relative activities when testosterone hydroxylation activity by using mediums containing 1 μmol/L of hydrocortisone equals 100%.

Three samples per condition were measured independently, and the mean values are shown. The standard deviations are indicated by error bar.

From these results, the less the number of hydroxy group becomes, the less the testosterone hydroxylation activities.

### Information on Donors of Hepatocytes

Donor 1

age: 67

race: Caucasian

sex: female

height: 163 cm (5′4″)

weight: 69 kg (151.8 lb)

date of hepatocyte preparation: Jan. 13, 1997

designation: HH-018

Donor 2

age: 76

race: Caucasian

sex: female

height: 152 cm (5′)

weight: 63.3 kg

date of hepatocyte preparation: unknown

designation: HH-022

Donor 3

age: 2.5

race: African American

sex: male

height: 94 cm (37.0″)

weight: 15 kg

date of hepatocyte preparation: May 28, 1997

designation: HH-029

Donor 4

age: 34

race: Caucasian

sex: male

height: 170 cm (67″)

weight: 100 kg

date of hepatocyte preparation: Nov. 4, 1999

designation: HH-110

Donor 5

age: 58

race: Caucasian

sex: male

height: 178 cm (5′10″)

weight: 79 kg (175 lb)

date of hepatocyte preparation: Mar. 6, 2000

designation: HH-118

Donor 6

age: 57

race: Hispanic

sex: male

height: 175 cm (5′9″)

weight: 85 kg

date of hepatocyte preparation: Aug. 8, 2000

designation: HH-135

Donor 7

age: 63

race: Caucasian

sex: male

height: 175 cm (5′9″)

weight: 93 kg

date of hepatocyte preparation: Dec. 18, 2000

designation: HH-148

Donor 8

age: 58

race: Caucasian

sex: male

height: unknown

weight: unknown

date of hepatocyte preparation: unknown

designation: IVT-077

Donor 9

age: 84

race: Caucasian

sex: female

height: unknown

weight: unknown

date of hepatocyte preparation: unknown

designation: IVT-088

Donor 10

age: 74

race: Caucasian

sex: female

height: unknown

weight: unknown

date of hepatocyte preparation: unknown

designation: IVT-100

Donor 11

age: 55

race: Caucasian

sex: female

height: unknown

weight: unknown

date of hepatocyte preparation: unknown

designation: IVT-124

Donor 12

age: 44

race: Caucasian

sex: female

height: unknown

weight: unknown

date of hepatocyte preparation: unknown

designation: XEN-254

### INDUSTRIAL APPLICABILITY

The method for determining the metabolic function of xenobiotics and induction thereof using the cryopreserved primary human hepatocytes, namely a technique for determining the enzyme activity and the gene expression thereof, involved in xenobiotic metabolism, and induction of the enzyme activity and induction of gene expression thereof, involved in xenobiotic metabolism, is useful for screening for a compound or a salt thereof, for example, that inhibits or promotes the enzyme activity and gene expression, involved in xenobiotic metabolism in the liver, and induction of the enzyme activity and the gene expression involved in xenobiotic metabolism in the liver, and for studying on the effects of a compound containing a pharmaceutical or candidate pharmaceutical compound on the metabolic function of xenobiotics in the liver. Further, the present invention allows us to examine the cells from different donors by same method and to determine the effects of a test compound on the cells from plural different donors simultaneously under the same conditions, and individual difference in the enzyme activity and the gene expression involved in xenobiotic metabolism in the liver, and induction of the activity and gene expression of an enzyme involved in xenobiotic metabolism in the liver can be investigated.

1. A method for assaying the function of a test compound to metabolize xenobiotics or the induction thereof which comprises contacting the test compound with hepatocytes maintained in a serum-free synthetic medium containing glucocorticoid, wherein the hepatocytes are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism.

2. The method according to claim 1, wherein glucocorticoid is hydrocortisone, dexamethasone or a mixture thereof.

3. The method according to claim 1, wherein glucocorticoid is hydrocortisone.

4. The method according to any one of claims 1 to 3, wherein the enzyme is UDP-glucuronyl transferase, flavin-containing monooxygenase, epoxide hydrolase, sulfotransferase, glutathione S-transferase, NADPH-cytochrome P450 reductase or cytochrome P450.

5. The method according to claim 4, wherein cytochrome P450 is CYP1A1, CYP1A2, CYP2A6, CYP2B6, CYP2C8, CYP2C9, CYP2C19, CYP2D6, CYP2E1, CYP3A4, CYP3A5 or CYP3A7.

6. The method according to any one of claims 1 to 3, wherein the activity of the enzyme is that of UDP-glucuronyl transferase, flavin-containing monooxygenase, epoxide hydrolase, sulfotransferase, glutathione S-transferase, NADPH-cytochrome P450 reductase, methoxyresorfin dealkylation, ethoxyresorfin dealkylation, pentoxyresorfin dealkylation, benzyloxyresorfin dealkylation, ethoxycoumarinresorfin dealkylation, coumarin hydroxylation, taxol hydroxylation, tolbutamide hydroxylation, (S)-mephenytoin hydroxylation, bufuralol hydroxylation, nitrophenol hydroxylation or testosterone hydroxylation.

7. The method according to any one of claims 1 to 3 which measures the enzyme activity or the gene expression, involved in xenobiotic metabolism.

8. The method according to any one of claims 1 to 3 which measures the mechanism for inducing the enzyme activity or the mechanism for inducing the gene expression, involved in xenobiotic metabolism.

9. The method according to any one of claims 1 to 3, wherein the serum-free synthetic medium further comprises one or more components selected from the group consisting of prolactin, cholera toxin and liver cell growth factor.

10. A method for maintaining (i) the enzyme activity or the gene expression, involved in xenobiotic metabolism, or (ii) the mechanism for inducing the enzyme activity or the mechanism for inducing the gene expression, involved in xenobiotic metabolism of hepatocytes, which comprises culturing cryopreserved primary human hepatocytes in a serum-free synthetic medium containing glucocorticoid after the hepatocytes are thawed.

11. The method according to claim 10, wherein glucocorticoid is hydrocortisone, dexamethasone or a mixture thereof.

12. The method according to claim 10, wherein glucocorticoid is hydrocortisone.

13. The method according to any one of claims 10 to 12, wherein the serum-free synthetic medium further comprises one or more components selected from the group consisting of prolactin, cholera toxin and liver cell growth factor.

14. Hepatocytes maintained by the method according to any one of claims 10 to 12.

15. A serum-free synthetic medium for culturing cryopreserved primary human hepatocytes after thawing which comprises glucocorticoid, prolactin, cholera toxin and liver cell growth factor.

16. The serum-free synthetic medium according to claim 15, wherein glucocorticoid is hydrocortisone, dexamethasone or a mixture thereof.

17. The serum-free synthetic medium according to claim 15, wherein glucocorticoid is hydrocrotisone.

18. A method for screening for a compound or a salt thereof that inhibits or enhances (i) the enzyme activity or the gene expression, involved in xenobiotic metabolism in the liver, or (ii) the mechanism for inducing the enzyme activity or the mechanism for inducing the gene expression, involved in xenobiotic metabolism in the liver, which comprises using the method according to any one of claims 1 to 3.

19. A compound or a salt thereof that inhibits or enhances (i) the enzyme activity or the gene expression, involved in xenobiotic metabolism in the liver, or (ii) the mechanism for inducing the enzyme activity or for the mechanism inducing the gene expression, involved in xenobiotic metabolism in the liver, which is obtained by the screening method according to claim 18.

20. A pharmaceutical composition comprising the compound or the salt thereof according to claim 19.

21. A method for determining the effect of a test compound on the function of the liver to metabolize xenobiotics, which comprises using the method according to any one of claims 1 to 3.

22. Use of a serum-free synthetic medium containing glucocorticoid for assaying the function of a test compound to metabolize xenobiotics or the induction thereof by contacting the test compound with hepatocytes which are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism.

23. Use of glucocorticoid for preparing a serum-free synthetic medium which is used for assaying the function of a test compound to metabolize xenobiotics or the induction thereof by contacting the test compound with hepatocytes which are obtained by thawing cryopreserved primary cultured human hepatocytes and retain (i) the enzyme activity or the gene expression, involved in xenobiotic metabolism, or (ii) the mechanism for inducing the enzyme activity or for inducing the gene expression, involved in xenobiotic metabolism.

24. Use of hepatocytes which are obtained by thawing cryopreserved primary human hepatocytes and retain (i) the enzyme activity or the gene expression, involved in xenobiotic metabolism, or (ii) the mechanism for inducing the enzyme activity or for inducing the gene expression, involved in xenobiotic metabolism, for assaying the function of a test compound to metabolize xenobiotics or the induction thereof by contacting the test compound with the hepatocytes maintained in a serum-free synthetic medium containing glucocorticoid.
